SEQUENCE LISTING (13 sequences)

SEQ ID NO: 1 - protein, 7 aa, Artificial Sequence
  Synthetic
FTTFTVT

SEQ ID NO: 2 - protein, 20 aa, Artificial Sequence
  Synthetic
DGIWKASFTTFTVTKYWFYR

SEQ ID NO: 3 - protein, 178 aa, Artificial Sequence
  Synthetic
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 4 - protein, 17 aa, Artificial Sequence
  Synthetic
NYHYLESSMTALYTLGH

SEQ ID NO: 5 - protein, 20 aa, Artificial Sequence
  Synthetic
WGIDKAFFTTSTVTYKWFRY

SEQ ID NO: 6 - protein, 16 aa, Artificial Sequence
  Synthetic
RQIKIWFQNRRMKWKK

SEQ ID NO: 7 - protein, 16 aa, Artificial Sequence
  Synthetic
RQIKIFFQNRRMKWKK

SEQ ID NO: 8 - protein, 16 aa, Artificial Sequence
  Synthetic
RQIKIWFQNRRMKFKK

SEQ ID NO: 9 - protein, 16 aa, Artificial Sequence
  Synthetic
RQIKIFFQNRRMKFKK

SEQ ID NO: 10 - protein, 27 aa, Artificial Sequence
  Synthetic
GWTLNSAGYLLGKINLKALAALAKKIL

SEQ ID NO: 11 - protein, 21 aa, Artificial Sequence
  Synthetic
KETWWETWWTEWSQPKKKRKV

SEQ ID NO: 12 - protein, 12 aa, Artificial Sequence
  Synthetic
  misc_feature (1)..(12): residues 1-12 of SEQ ID NO11
KETWWETWWTEW

SEQ ID NO: 13 - protein, 6 aa, Artificial Sequence
  Synthetic
  misc_feature (6)..(6): the 6 C-term residues of SEQ ID NO11
KKKRKV

(19) United States
(12) Patent Application Publication
(10) Pub. No.: US 2022/0370544 A1
(43) Pub. Date: Nov. 24, 2022

(54) PEPTIDE THERAPEUTICS FOR ACUTE AND CHRONIC AIRWAY AND ALVEOLAR DISEASES

(71) Applicant: BOARD OF REGENTS, THE UNIVERSITY OF TEXAS SYSTEM, Austin, TX (US)

(72) Inventor: Sreerama SHETTY, Tyler, TX (US)

(73) Assignee: BOARD OF REGENTS, THE UNIVERSITY OF TEXAS SYSTEM, Austin, TX (US)

(21) Appl. No.: 17/615,524

(22) PCT Filed: Nov. 21, 2019
(86) PCT No.: PCT/US2019/062543

§ 371 Date: Nov. 30, 2021

Related U.S. Application Data
(60) Provisional application No. 62/770,508, filed on Nov. 21, 2018.

(51) Int. Cl. A61K 38/08; A61P 11/00

(52) U.S. Cl. CPC A61K 38/08; A61P 11/00

(57) ABSTRACT

Chronic obstructive pulmonary disease (COPD) is a chronic inflammatory disease encompassing chronic bronchitis, emphysema and remodeling of small airways that can be treated by the caveolin-1 peptide CSP7 (SEQ ID NO:1). Chronic tobacco smoke exposure (TSE)-induced lung injury includes increased alveolar and airway inflammation, type II alveolar epithelial cells (A₂Cs) senescence and apoptosis, and mucus hypersecretion by AECs. Interleukin 17A-mediated induction of plasminogen activator inhibitor-1 (PAI-1) expression through caveolin-1 led to TSE-induced lung injury, which was abrogated by CSP7 treatment which abolished A₂Cs senescence and apoptosis, and AECs mucus hypersecretion in TSE wild type (WT) mice. Ex vivo CSP7 treatment of lung tissue of COPD patients decreased A₂C apoptosis and AEC mucus hypersecretion. Lung injury induced by PAI-1 expression in COPD lung tissue and WT mice (20 weeks TSE), with A₂Cs senescence and apoptosis, and AEC mucus hypersecretion was abolished by CSP7.

### BACKGROUND OF THE INVENTION

### Field of the Invention

The present invention in the field of biochemistry and medicine is directed to methods and composition for increasing lung cell viability through inhibition of senescence and apoptosis, reducing mucin, interleukin 17A (IL-17A), p53 and Plasminogen activator inhibitor-1 (PAI-1) and increasing urokinase plasminogen activator (uPA), uPA receptor (uPAR) and expression of the gene of the forkhead family, FOXA1 (which encodes Hepatocyte nuclear factor 3-α) in airway and alveolar epithelial cells and reducing smooth muscle activation, and for treating chronic obstructive pulmonary disease (COPD)/emphysema, severe asthma, α1 anti-trypsin deficiency, cystic fibrosis, bronchiectasis, sarcoidosis, bronchiolitis obliterans, lung allograft fibrogenesis and lung transplant rejection,

### Description of the Background Art

COPD affects up to 24 million people and is the third leading cause of death in the U.S. (Hurd S, Chest, 2000; 117: 1S-4S; Ford E S et al., Chest, 2013; 144: 284-305). Chronic tobacco smoke exposure (TSE) is a major risk factor for COPD. There are currently no interventions available to reverse the progression of COPD-related lung injury. Acute exacerbations of COPD are the second leading cause of hospital stays and incur costs of >$18 billion annually in the US (Ford E S, et al., Chest, 2015; 147: 31-45.

Airway epithelial cells (AECs) and alveolar type II epithelial cells (A₂Cs) are common targets for damage from TSE and from mediators/cytokines released from inflammatory cells. COPD pathogenesis has been directly linked to a loss of alveolar structure due to A₂C senescence and apoptosis (Shetty S K et al., Am J Respir Cell Mol Biol. 2012; 47:474-83; Park J-W, et al., COPD. 2007; 4:347-53; Tsuji T et al., Am J Respir Cell Mol Biol. 2004; 31:643-49). Further, TSE causes airway inflammation and mucus hypersecretion leading to airway plugging. The reports of the present inventor and colleagues (Shetty S K et al., supra; Bhandary Y P et al., PLoS One. 2015; 10: e0123187; Tiwari N et al., Am J Physiol Lung Cell Mol Physiol. 2016; 310:L496-506; Marudamuthu A S et al., Am J Pathol. 2015; 185: 55-68; Shetty S et al., J Biol Chem. 2008; 283: 19570-80; Bhandary Y P et al., Toxicol Appl Pharmacol. 2015; 283: 92-98; Shetty S K et al., Am J Pathol. 2017; 187:1016-34) and their preliminary data indicate that TSE lung injury primarily involves increased alveolar and airway inflammation, A₂Cs senescence and apoptosis, and mucus hypersecretion by AECs. These changes are intricately linked to induction of p53 and Plasminogen activator inhibitor-1 (PAI-1), telomere dysfunction in A₂Cs, and mucus cell metaplasia and overexpression of the Mucin 5AC (MucAC or M5Ac) gene/protein by AECs and all are clinically relevant and occur in COPD patients. Supporting this point, the present inventors' findings and publications using A₂Cs and AECs, or lung sections of COPD patients and mouse model of TSE lung injury link these findings. These studies showed that p53-mediated induction of PAI-1 expression in A₂Cs and AECs augmented lung inflammation and A₂Cs senescence and apoptosis, mucus hypersecretion in AECs and predisposed to respiratory infection, which often occurs in COPD. Further, a deficiency in p53 or PAI-1 leaves mice resistant to TSE lung injury (Shetty S K et al., supra; Bhandary et al, supra)

p53-Induced PAI-1 Expression, Alveolar Fibrinolysis and A₂Cs Apoptosis:

Lung lavage fluids exhibit high levels of urokinase-type plasminogen activator (uPA) activity and contribute to alveolar proteolysis (Idell S et al., J Clin Invest. 1989; 84: 695-705; Barazzone C et al., J Clin Invest. 1996; 98:2666-73; Olman M A et al., J Clin Invest. 1995; 96:1621-30). However, impaired fibrinolysis is mainly attributable to local over-expression of PAI-1 (major inhibitor of uPA) injury (Barazzone et al., supra; Olman et al., supra; Chapman H A et al., Am Rev Respir Dis. 1986; 133:437-43; Chapman H A. J Clin Invest. 2004; 113:148-57; Hasday J D et al., Exp Lung Res. 1988; 14: 261278; Bertozzi P et al., N Engl J Med. 1990; 322: 890-97; Bachofen M et al., Clin Chest Med. 1982; 3:35-56; Idell S et al., supra; Eitzman D T et al., J Clin Invest. 1996; 97:232-37; Lardot C G et al., Am J Respir Crit Care Med. 1998; 157:617-28; Xu X et al., Exp Lung Res. 2009; 35:795-805; Hu X et al., Chin Med J (Engl). 2009; 122: 2380-85; Zidovetzki R et al., Stroke J Cereb Circ. 1999; 30:651-55).

p53, by binding through its C-terminal amino acid residues 296-393 with a 70-nucleotide (nt) destabilization determinant of PAI-1 3′UTR mRNA (p53Bp) has been shown to induce PAI-1 (Shetty, S, 2008, supra; Shetty P et al., Am J Respir Cell Mol Biol. 2008; 39:364-72; Shetty S et al., Mol Cell Biol. 2007; 27:5607-18). p53 also binds PAI-1 promoter and increases PAI-1 mRNA transcription (Kunz C et al., Nuc Acids Res. 1995; 23:3710-17; Bhandary Y P et al., Am J Pathol. 2013; 183:131-43). The present inventors and colleagues further found that TSE of A₂Cs and AECs increased p53 and PAI-1 expression, and reduced cell viability, which was reversed by inhibition of p53 binding to endogenous PAI-1 mRNA, and tissues from COPD patients also showed elevated p53 and PAI-1 in A₂Cs (Shetty S K, 2012, supra; Bhandary Y P et al., PLoS One. 2015, supra; Tiwari et al., supra; Marudamuthu A S et al., supra).

Role of IL-17A in TSE Lung Injury:

Studies in COPD patients revealed that the accumulation of pulmonary lymphoid follicles and IL-17A⁺ mast cells were associated with severe COPD (Roos A B et al., Am J Respir Crit Care Med. 2015; 191:1232-41. These cells secreted IL-17A, which then set up an inflammatory positive feedback loop as well as MMP12, a potent enzyme that predisposes to emphysema. The present inventors' preliminary findings (Tiwari N et al., supra) and recent reports (Zou Y et al., Int J Chron Obstruct Pulmon Dis. 2017; 12:1247-1254; Chang Y et al., Respir Res. 2014; 15:145) revealed that IL-17A was markedly elevated in the lung and sputum of COPD patients. IL-17A levels were significantly increased in TSE mice, while those mice lacking IL-17A resisted TSE injury. According to the present invention, IL-17A augments p53 and PAI-1 in A₂Cs. Further, literature suggested that IL-17A promoted mucus cell metaplasia and M5Ac overexpression (Xia W et al., PLoS ONE. 2014; 9)

According to the present invention, IL-17A, p53 and PAI-1 affect TSE-induced telomere dysfunction in A₂Cs and emphysema, and MSAc overexpression by AECs and airway/lung remodeling.

In summary, AECs and A₂Cs are the common targets of damage from chronic TSE and inflammatory cells in humans and in pre-clinical COPD models. COPD and TSE lung injury is also characterized by lung inflammation, telomere dysfunction, and senescence and apoptosis in A₂Cs and MSAc overexpression by AECs.

The present inventors and colleagues have linked these findings, showing that A₂Cs and AECs express p53 and PAI-1, and that p53 induces PAI-1 to increase lung injury (references cited above and Eren M et al., Proc Natl Acad Sci USA. 2014; 111: 7090-95 and Bhandary Y P et al., Am J Physiol Lung Cell Mol Physiol. 2012; 302: L463-73).

A deficiency of p53 or PAI-1 makes mice highly resistant to TSE lung injury (supra), implying that changes in p53 and PAI-1 in A₂Cs and AECs, and consequent telomere dysfunction, alveolar injury and mucus hypersecretion are important contributors to COPD. Our data further suggest that TSE or IL-17A augments p53 and PAI-1 expression, and the process involves increased Cav-1. According to the present invention, airway delivery of the heptapeptide CSP7 (described below) in liquid or DP formulation mitigates deleterious effects of TSE.

Telomere Failure

A subset of patients with age-associated pathology such as idiopathic pulmonary fibrosis (IPF) manifests mutations in the gene of telomerase reverse transcriptase (TERT), or in its RNA component (TERC) (Armanios M Y et al., N Engl J Med 2007; 356:1317-26; Tsakiri K D et al., Proc Natl Acad Sci USA 2007; 104:7552-57) the mutation in TERT can be familial as well as non-familial (Tsakiri et al., supra), suggesting that factors contributing to mutations can in turn affect telomere failure and associated pathologies.

The mechanisms by which telomere defects provoke lung disease are not understood, but a number of observations have pointed to lung-intrinsic factors and epithelial dysfunction as candidate events (Alder J K et al., Proc Natl Acad Sci USA 2015; 112:5099-5104). For example, in telomerase-null mice, DNA damage preferentially accumulates in the air-exposed epithelium after environmentally induced injury, such as with cigarette smoke. The additive effect of environmental injury and telomere dysfunction has been suggested to contribute to the susceptibility to emphysema seen in these mice (Alder et al., supra). Pulmonary fibrosis and emphysema patients have also been noted to have abnormally short telomeres in AEC2s (Liu T et al., Am J Respir Cell Mol Biol 2013; 49:260-68).

As noted, in COPD/emphysema, chronic inflammation leads to muc5A overexpression and mucus hypersecretion AECs, narrowing of small airways due to inflammation and airway remodeling and smooth muscle proliferation, and alveolar wall destruction due to death of A₂Cs. This is also true in wild-type (WT) mice exposed to 20 wks of tobacco smoke. Further, levels of the cytokine Interleukin-17A (IL-17A) are significantly elevated in the peripheral lung tissues of patients with severe COPD (and in the lungs of TSE mice (genetically WT).

Shortening of the telomere due to increased expression of SIAH-1, a p53-inducible E3 ubiquitin ligase that is known to downregulate the telomere repeat binding factor 2 (TRF2) was observed in A₂Cs of COPD patients. Downregulation of telomerase reverse transcriptase (TERT) was observed, and was correlated with the reduced TRF2 and upregulation of TRF1 expression in the COPD lung tissues.

The additive effect of environmental injury and telomere dysfunction has been suggested to contribute to the susceptibility to emphysema (Alder et al. Am J Crit Care Med 184: 904-912, 2011). In emphysema patients, telomeres in A₂Cs are abnormally short. This is also true in A₂Cs from WT mice subjected to tobacco smoke. However, the mice exposed to smoke and received caveolin-1 scaffolding domain peptide; CSP7, resisted telomere shortening. Increase in the protein expression of p53, cleaved caspase-3 and β-galactosidase, pointing to A₂C death. However, the A₂Cs from the CSP7 treated mice showed significant decreases in p53, cleaved caspase-3 and β-galactosidase expression. CSP7 treatment also restored TRF2 expression and the enzyme activity of TERT.

There is presently no cure or effective treatment intervention for mucus hypersecretion, telomere shortening associated with COPD/emphysema and bronchiolitis obliterans associated with transplant rejection. In view of the poor prognosis and lack of therapeutic approaches for these conditions, there is an urgent need for new interventions to reverse or at least slow the progression of disease. This critical therapeutic gap is addressed by the present invention.

Airway mucus hypersecretion is one of the cardinal features of several chronic lung diseases including COPD, which results in airway obstruction and contributes significantly to morbidity and mortality (Hogg J C et al., N Engl J Med 350: 2645-53, 2004; Hogg J C et al., Annu Rev Pathol 4: 435-59, 2009). Clinically, muco-active drugs have been shown to effectively reduce exacerbation of COPD and improve to upsurge the quality of life of patients (Curran D R et al., Am J Respir Cell Mol Biol 42:268-75, 2010; Decramer M et al., Eur Respir Rev 19:134-40, 2010), demonstrating the usefulness of targeting mucus hypersecretion in COPD therapy. Chronic TSE is the most common identifiable risk factor for COPD, with smokers known to have a greater COPD mortality rate than non-smokers (Kohansal R et al., Am J Respir Crit Care Med.; 180:3-10, 2009). The pathogenesis of COPD remains poorly understood but involves aberrant cellular and inflammatory responses of the lung to TSE, resulting in the disruption of airway epithelial cell (AEC) function. Such disruption has been attributed to a reduction in epithelial cell cilia length and AEC death, followed by re-epithelialization by goblet cells, subsequent excess mucus production finally leading to impaired mucociliary clearance.

In total, 21 genes are reported to encode mucins in the human genome. Mucin 5Ac (MUCSAC) is expressed at high levels in the airway system (Thornton D J et al., Annu Rev Physiol 70: 459-86, 2008; Rose M C et al., Physiol Rev 86: 245-78, 2006). Mucus may alter the normal structure and status of goblet cells after failing to incorporate with MUCSAC. Without the normal reaction between MUCSAC and mucus, the airway viscoelasticity becomes vulnerable to plugging (Bonser L R et al., J Clin Med 6: E112, 2017; Woodruff P G et al., Am J Respir Crit Care Med 180:388-95, 2009). Goblet cell differentiation is dictated by a large network of genes, in which transcription factors sterile α motif-(SAM)-pointed domain containing ETS-like transcription factor (SPDEF) and forkhead box protein A2 (FOXA2) are two key regulators. SPDEF (encoded in humans by the SPDEF gene; Genbank Gene ID 25803) is required for goblet cell differentiation and mucus production, including the major secreted airway mucin MUCSAC (Park K S et al., J Clin Invest. 117:978-88, 2007; Chen G et al., J Clin Invest 119:2914-24, 2009; Rajavelu P et al., J Clin Invest. 125:2021-31, 2015), whereas FOXA2 is a potent inhibitor of goblet cell differentiation in the lung (Wan H et al., Development. 131:953-64, 2004; Chen G et al., J Immunol. 184:6133-41 2010; Tang X et al., Am J Respir Cell Mol Biol. 49:960-70, 2013). Forkhead box protein A₃ (FOXA3) was highly expressed in airway goblet cells from COPD patients. Because FOXA3 bound to and induced SPDEF, a gene required for goblet cell differentiation in the airway epithelium, the observed effects of FOXA3 on mucus-related gene expression are likely mediated, at least in part, by its ability to induce SPDEF (et al., Am J Respir Crit Care Med. 2014 Feb. 1; 189:301-13).

Breakdown of the ciliated cells also further contributes to mucociliary dysfunction. Epithelial cells exposed to TSE have an over 70% decrease in the number of ciliated cells and show a shortening of the cilia. One mechanism under investigation involves autophagy that is dependent on histone deacetylase 6 (HDAC6). HDAC6 is upregulated in the airways of COPD patients where it may act to target damaged and misfolded proteins for proteasomal degradation. In the case of ciliary shortening, HDAC6 was found to co-localize with alpha tubulin then associated with LC3B, a protein active in autophagy. Recently demonstrated an increased expression of autophagy markers in the development of COPD (Kim H P et al., Autophagy, 4:887-95, 2008; Ryter S W et al., Autophagy 5:235-7, 2009).

Caveolae are vesicular invaginations of the plasma membrane. Caveolin-1 is the structural protein component of caveolae. Caveolin-1 participates in signal transduction processes by acting as a scaffolding protein that concentrates, organizes and functional regulates signaling molecules within caveolar membranes. These studies, combined with the close association between MUCSAC secretion and airway inflammation, led us to hypothesize that caveolin-1 may be an important regulator involved in TSE-induced MUCSAC production in lung epithelial cells. Currently, few advances have been made to alleviate MCC disruption and bronchitis associated with the pathogenesis of COPD due to elevation of caveolin1. In the present study, we investigated Caveolin 1 bind to the catalytic unit (PP2AC) of protein phosphatase-2A (PP2A), which in turn downregulated PP2AC activity and led to increased expression of cancerous inhibitor of protein phosphatase 2A (CIP2A). Increased CIP2A leads to phosphorylation of the serine/threonine-selective protein kinase (ER)K, and secretion of matrix metalloproteinase-12 (MMP12). Indeed, caveolin 1 elevated p53 and PAI-1 expression in AECs and increased susceptibility to and exacerbation of respiratory infections which all are associated with COPD.

According to the present invention, caveolin-1 as a key player of a novel signaling pathway that links TSE to mucus hypersecretion and ciliary disassembly. A 7-mer deletion fragment of caveolin-1 scaffolding domain peptides CSP referred to as CSP7 (having the sequence FTTFTVT (SEQ ID NO:1) mitigates cilia shortening and impaired mucociliary clearance (MCC) by inhibiting caveolin-1. These findings provide both new insights on how CSP7 regulates complex interrelationships between p53, PAI-1, autophagy and primary cilia, but also provides a basis for treatment of ciliopathy-associated mucus hypersecretion. The present results provide new therapeutic targets for improving airway function during chronic lung diseases such as COPD through the maintenance of epithelial cell proteostasis and modulation of the autophagic pathway.

### Caveolin-1-Derived Peptides

The present inventors first discovered that a 20 residue peptide DGIWKASFTTFTVTKYWFYR, SEQ ID NO:2) which is the scaffolding domain of caveolin-1 (Cav-1; SEQ ID NO:3, shown below) protected lung or airway epithelial cells (LECs/AECs) from bleomycin (“BLM”)-induced apoptosis in vitro and in vivo and prevented subsequent pulmonary fibrosis by attenuating lung epithelial damage (Shetty et al., U.S. patent application Ser. No. 12/398,757 published as U.S. 2009-0227515A1 (Sep. 10, 2009) and issued as U.S. Pat. No. 8,697,840 (Apr. 15, 2014)) and Shetty et al., PCT Pub. WO2014/145389 (Sep. 18, 2014), corresponding to U.S. application Ser. No. 14/775,895 published as U.S. Pat. Publ. 2016/0272678 (Sep. 22, 2016) and issued as U.S. Pat. No. 9,630,990 (Apr. 25, 2017), all of which are hereby incorporated by reference in their entirety.

The present inventors also discovered that a 17 residue peptide NYHYLESSMTALYTLGH (SEQ ID NO:4), termed PP-2, also protected LECs from BLM-induced apoptosis in vitro and in vivo and prevented subsequent pulmonary fibrosis by attenuating lung epithelial damage.

Shetty et al., 2009 and 2014 (supra) also describes biologically active substitution, addition and deletion variants of these peptides as well as peptide multimers and deliverable polypeptides comprising the above peptides, and pharmaceutical compositions comprising the foregoing peptides, variants and multimers. Those compositions inhibit apoptosis of injured or damaged lung epithelial cells and treating acute lung injury and consequent pulmonary fibrosis/IPF.

Shetty et al. 2014 (U.S. Pat. No. 9,630,990) identified a particular 7 residue fragment of CSP now termed CSP7, which has the sequence FTTFTVT (SEQ ID NO:1) and which has the biological activity of CSP. More recently the present inventors' group has described formulations of CSP7 as an inhaled peptide therapeutic for, inter alia, idiopathic pulmonary fibrosis (Surasaranga et al., Drug Devel. Indust. Pharmacy, 2018; 44:184-98) which peptide is also used in the present methods. The present invention constitutes, in part, an extension of the inventors' earlier findings as disclosed in the above patents and patent publications (S. Shetty et al., 2007, 2008 & 2009, 2014, supra).

### SUMMARY OF THE INVENTION

The present invention is directed to methods using the heptapeptide CSP7 (FTTFTVT, SEQ ID NO:1) which is the smallest functional fragment of the 20 residue peptide DGIWKASFTTFTVTKYWFYR (SEQ ID NO:2) which is the scaffolding domain (CSP or CSP1) of caveolin-1 (Cav-1).

CSP7 blocks, inhibits, attenuates or reduces
induction of p53 and PAI-1,
telomere shortening and dysfunction,
senescence and apoptosis in alveolar type II epithelial cells (A₂Cs),
expression of forkhead box protein A₃ (FOXA3)
expression of sterile α motif-(SAM)—pointed domain containing ETS-like factor (SPDEF)
expression cancerous inhibitor of protein phosphatase 2A (CIP2A);
expression of histone deacetylase 6 (HDAC6),
mucus cell metaplasia,
mucus hypersecretion and M5Ac overexpression such as that induced by tobacco smoke in AECs,
airway remodeling,
IL-17A and IL-17A-mediated mucus hypersecretion and resultant lung inflammation,
epithelial cell injury,
autophagic activity,
airway ciliary disassembly, shortening or ciliopathy,
transplant rejection and
lung allograft fibrogenesis.
   CSP7 inhibits tobacco-smoke-induced muc5A expression by AECs and telomere shortening by suppressing p53-miR-34a feed-forward induction and protecting sheltrin complex proteins in A₂Cs.

CSP7 increases expression of, or upregulates:
forkhead A₂ (FOXA2)
catalytic unit (of protein phosphatase-2A (PP2AC).

Therefore, CSP7, preferably in a formulation for administration by inhalation/lung instillation as described herein (see, also, Surasaranga et al., supra) is an effective agent for treating inflammatory lung diseases such as COPD/emphysema, severe asthma, al anti-trypsin deficiency, cystic fibrosis, sepsis, bronchiectasis, sarcoidosis and other airway diseases. Since increased IL-17A contributes to bronchiolitis obliterans, inhibition of IL-17A by treatment with CSP7 reduces or prevents transplant rejection including that stimulated by or resulting from allograft fibrogenesis.

The present invention is directed to a method for

(A) blocking, reducing or attenuating:
induction of p53 and PAI-1;
(ii) telomere dysfunction;
(iii) senescence and apoptosis in A₂Cs;
(iv) expression of FOXA3
(v) expression of SPDEF
(vi) mucus cell metaplasia;
(vii) mucus hypersecretion mediated by overexpression of M5Ac or by IL-17A by AECs;
(viii) expression CIP2A;
(ix) expression of HDAC6;
(x) autophagic activity; or
(xi) ciliary disassembly, shortening or ciliopathy;
or
   (B) increasing expression of or upregulation of:
(xii) expression of forkhead box protein A₂ (FOXA2);
(xiii) expression of catalytic unit of protein phosphatase-2A (PP2AC);
   comprising providing to A₂Cs or AECs in a subject, preferably a human subject, an effective amount of a compound or composition that is:
(a) a peptide designated CSP7 the sequence of which is FTTFTVT (SEQ ID NO:1);
(b) an addition variant of (a) that includes 1-5 amino acids of additional sequence at the N- and/or C-terminus
(c) a covalently-modified chemical derivative of the peptide of (a) or (b),
(d) a peptide multimer of (a), (b) or (c);
(e) a deliverable peptide or polypeptide composition comprising the peptide, variant derivative or multimer of any of (a)-(d) bound to or associated with a delivery or translocation-molecule or moiety;
wherein said variant, chemical derivative or multimer has at least 20% of the biological or biochemical activity of said CSP7 in an in vitro or in vivo assay.

The above method preferably results in a reduction of lung inflammation and treatment, attenuation or reduction of an inflammatory lung disease in said subject.

The peptide variant, chemical derivative or multimer described above or below preferably has the following activity relative to the activity CSP7: at least about 20%, 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%. The peptide variant chemical derivative or multimer may have 100% or greater than 100% of the activity of CSP7. This relative activity may be based on any method disclosed herein or known in the art for evaluating such activity.

A preferred compound is the heptapeptide CSP7, FTTFTVT (SEQ ID NO:1).

A preferred peptide multimer comprises at least two monomers, each monomer being the CSP7 peptide, the variant of (b) above or the chemical derivative of (c) above, which multimer:
(a) has the formula P¹ₙ wherein
   (i) P¹ is the peptide, variant or chemical derivative as above, and
   (ii) n=2-5, or
(b) has the formula (P¹-Xₘ)ₙ-P², wherein
   (i) each of P¹ and P² is, independently, the peptide, variant of chemical derivative as above,
   (ii) each of P¹ and P² is the same or different peptide, variant or derivative
   (iii) X is C₁-C₅ alkyl, C₁-C₅ alkenyl, C₁-C₅ alkynyl, C₁-C₅ polyether containing up to 4 oxygen atoms;
   (iv) m=0 or 1; and
   (v) n=1-7,
(c) has the formula (P¹-Gly_{z})ₙ-P², wherein:
   (i) each of P¹ and P² is, independently, the peptide, variant or derivative,
   (ii) each of P¹ and P² is the same or different peptide or variant or derivative;
   (iii) z=0-6; and
   (iv) n=1-25,

The peptide multimer preferably has at least 20% of the biological, biochemical or pharmacological activity of the CSP7 peptide in an in vitro or in vivo assay.

In the above method, the peptide, addition variant, chemical derivative, multimer, or deliverable peptide or polypeptide is provided in vivo.

Also provided is a method for treating a mammalian subject, preferably a human, having an inflammatory lung disease or condition, preferably selected from the group consisting of COPD/emphysema, severe asthma, al anti-trypsin deficiency, cystic fibrosis, bronchiectasis, sarcoidosis, bronchiolitis obliterans, lung allograft fibrogenesis and lung transplant rejection. The method comprises administering to the subject in need thereof and effective amount of
(a) a pharmaceutical composition comprising a compound or composition selected from the group consisting of:
   (i) a peptide designated CSP7 the sequence of which is FTTFTVT (SEQ ID NO:1);
   (ii) an addition variant of (i) that preferably does not exceed 20 residues and preferably includes 1-5 amino acids of additional sequence at the N-terminus, the C-terminus, or both;
   (iii) a covalently-modified chemical derivative of the peptide of (i) or (ii),
   (iv) a peptide multimer of (i), (ii) or (iii); and
   (v) a deliverable peptide or polypeptide composition comprising the peptide, variant derivative or multimer of any of (i)-(iv) bound to or associated with or admixed with a delivery or translocation-molecule or moiety.
   wherein said addition variant, chemical derivative or multimer has at least 20% of the biological, biochemical and pharmacological activity of said CSP7 in an in vitro or in vivo assay, and
(b) a pharmaceutically acceptable carrier or excipient.

In preferred embodiment of the above method, the compound is the CSP7 peptide of SEQ ID NO:1, In another embodiment of the method, the compound is the peptide multimer, preferably one that comprises monomers of the CSP7 peptide (SEQ ID NO:1).

Preferably, when the above method uses a peptide multimer:
(a) the peptide multimer has the formula P¹ₙ wherein
   (i) P¹ is the peptide, variant or chemical derivative, and
   (ii) n=2-5, or
(b) the peptide multimer has the formula (P¹-Xₘ)ₙ-P², wherein
   (i) each of P¹ and P² is, independently, the peptide, variant or chemical derivative;
   (ii) each of P¹ and P² is the same or different peptide, variant or derivative;
   (iii) X is C₁-C₅ alkyl, C₁-C₅ alkenyl, C₁-C₅ alkynyl, C₁-C₅ polyether containing up to 4 oxygen atoms;
   (iv) m=0 or 1; and
   (v) n=1-7, or*
(c) the peptide multimer has the formula (P¹-Gly_{z})ₙ-P², wherein:
   (i) each of P¹ and P² is, independently, the peptide, variant or derivative,
   (ii) each of P¹ and P² is the same or different peptide or variant or derivative;
   (iii) z=0-6; and
   (iv) n=1-25,

Preferably the multimer has at least 20% of the biological, biochemical or pharmacological activity of CSP7 peptide in an in vitro or in vivo assay.

The invention also provide a use of compound or composition for treating COPD/emphysema, severe asthma, al anti-trypsin deficiency, cystic fibrosis, bronchiectasis, sarcoidosis, bronchiolitis obliterans, lung allograft fibrogenesis and lung transplant rejection, which compound or composition comprises;

(a) a peptide designated CSP7 the sequence of which is FTTFTVT (SEQ ID NO:1);

(b) an addition variant of (a) that includes 1-5 amino acids of additional sequence at the N- and/or C-terminus;

(c) a covalently-modified chemical derivative of the peptide of (a) or (b),

(d) a peptide multimer of (a), (b) or (c); and

(e) a deliverable peptide or polypeptide composition comprising the peptide, variant derivative or multimer of any of (a)-(d) bound to or associated with a delivery or translocation-molecule or moiety.
wherein said addition variant, chemical derivative or multimer has at least 20% of the biological, biochemical or pharmacological activity of said CSP7 in an in vitro or in vivo assay.

Also provided is the use of a compound or composition for the manufacture of a medicament for treatment of COPD/emphysema, severe asthma, al anti-trypsin deficiency, cystic fibrosis, bronchiectasis, sarcoidosis, bronchiolitis obliterans, lung allograft fibrogenesis and lung transplant rejection, which compound or composition comprises:
(a) a peptide designated CSP7 the sequence of which is FTTFTVT (SEQ ID NO:1);
(b) an addition variant of (a) that includes 1-5 amino acids of additional sequence at the N- and/or C-terminus;
(c) a covalently-modified chemical derivative of the peptide of (a) or (b),
(d) a peptide multimer of (a), (b) or (c); and
(e) a deliverable peptide or polypeptide composition comprising the peptide, variant derivative or multimer of any of (a)-(d) bound to or associated with a delivery or translocation-molecule or moiety.
wherein said addition variant, chemical derivative or multimer has at least 20% of the biological or biochemical activity of said CSP7 in an in vitro or in vivo assay.

In embodiments of the foregoing method, the peptide, variant or chemical derivative is capped at its N-terminus, C-terminus or both with a capping group as described herein or otherwise known in the art.

In addition to the “standard” L-amino acids, D-amino acids or non-standard, modified or unusual amino acids which are well-defined in the art are also contemplated for use in the present invention for the purpose of protecting the peptide from proteolytic degradation in vivo.

### BRIEF DESCRIPTION OF THE DRAWINGS

FIG. 1 shows shortening of telomere length of A₂Cs obtained from human fibrotic lung. (A) TeloTAGGG assay was conducted for estimating the telomere length of the isolated genomic DNA. The southern blot data shows the telomere shortening of the A₂Cs from fibrotic lung. (B) Bar graph (PCR) shows the relative quantification of the shortening occurred in the A₂Cs. (C) Western blot analysis was conducted to analyze the protein expression of telomerase enzyme (TERT), and the apoptosis pathway related proteins. (D) Bar graph showing relative telomere length of the A₂Cs analyzed by qPCR after extracting the genomic DNA. E. Gel shows the telomerase enzyme activity as analyzed by the TRAPeze enzyme assay. (F) Bar graph shows the quantitation of the relative TRAPeze enzyme activity (n=2).

FIG. 2 shows shortening of telomere length of A₂Cs isolated from human COPD lungs. (A). TeloTAGGG assay was conducted for estimating the telomere length of the isolated genomic DNA. The southern blot data shows the telomere shortening of the AECs from COPD lung. (B) Bar graph (PCR) shows the relative quantification of the shortening occurred in the A₂Cs. C. Bar graph shows relative telomere length of the A₂Cs analyzed by qPCR after extracting the genomic DNA. (D) Western blot analysis was conducted to analyze the protein expression of telomerase enzyme (TERT), and the apoptosis pathway related proteins. (E) Gel shows the telomerase enzyme activity as analyzed by the TRAPeze enzyme assay. (F) Bar graph shows the quantitation of the relative TRAPeze enzyme activity (n=2).

FIG. 3 shows that passive cigarette smoke exposure led to decrease in telomerase expression and shortening of telomere in A₂Cs of WT mice. WT mice were exposed to smoke for 20 weeks, and then treated with peptide CSP7 or a control peptide (CP) and the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity (n=2).

FIG. 4 shows that repeated bleomycin exposure led to decrease in telomerase expression and shortening of telomere in A₂Cs of WT mice. WT mice were exposed to intranasal bleomycin once in two weeks for 16 weeks. CSP7 or control peptide (CP) treatment started at 14^{th} week and was continued daily till the end of the experiment, at which time A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the trapeze enzyme activity is shown (n=2).

FIG. 5 shows that A₂Cs of mice deficient in miR-34a expression were protected from telomere shortening induced by passive cigarette smoke. SP-CCRE-miR-34^{acKO} and SP-CCRE-miR-34a^{fl/fl} mice were exposed to smoke for 20 weeks and later treated with CSP7 or control peptide (CP) after which A₂Cs were isolated. (A) miR-34a expression by qPCR. (B) Relative telomere length of the A₂Cs was analyzed by qPCR after extracting the genomic DNA. (C) Western blot analysis was conducted to analyze the protein expression of telomerase enzyme (TERT), and the apoptosis pathway related proteins. (D) Gel shows the telomerase enzyme activity as analyzed by the TRAPeze enzyme assay. (E) Bar graph shows the quantitation of the TRAPeze enzyme activity (n=2).

FIG. 6 shows that passive cigarette smoke exposure led to decrease in telomerase expression and shortening of telomere in A₂Cs of uPA^{-/-} mice. uPA^{-/-} mice were exposed to smoke for 20 weeks and treated with the CSP7 or control peptide (CP) after which the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown (n=2).

FIG. 7 shows that repeated bleomycin exposure led to decrease in telomerase expression and shortening of telomere in A₂Cs of uPA^{-/-} mice. uPA^{-/-} mice were exposed to intranasal bleomycin once every two weeks for 16 weeks. CSP7 or control peptide (CP) treatment was started at 14^{th} week and continued daily till the end of the experiment at which time the A₂Cs were isolated. (A) Relative telomere length of the AECs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown (n=2).

FIG. 8. shows that A₂Cs of PAI-1^{-/-} mice were resistant to telomere shortening induced by passive cigarette smoke. PAI-1^{-/-} mice were exposed to smoke for 20 weeks, and then treated with the CSP7 or control peptide (CP) and the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity (n=2).

FIG. 9 shows that A₂Cs of PAI-1^{-/-} mice were resistant to telomere shortening induced by treatment with repeated dose of bleomycin. PAI-1^{-/-} mice were exposed to intranasal bleomycin once every two weeks for 16 weeks. CSP7 or control peptide (CP) treatment started at 14^{th} week and continued daily until the end of the experiment, at which time the A₂Cs were isolated. (A) Relative telomere length of the A₂Cs was analyzed by qPCR after extracting the genomic DNA. (B) Western blot analysis was conducted to analyze the protein expression of telomerase enzyme (TERT), and the apoptosis pathway related proteins. (C) Gel shows the telomerase enzyme activity as analyzed by the TRAPeze enzyme assay. (D) Bar graph shows the quantitation of the TRAPeze enzyme activity is shown (n=2).

FIG. 10 is a schematic illustration of how tobacco smoke exposure-induces airway mucus hypersecretion and ciliary disassembly and COPD and its attenuation by CSP7

FIG. 11A-11C show that differential expression of MUCSAC, FOXA2, FOXA3, HDAC6, and SPDEF in AECs isolated from COPD lungs. (A) bar graph showing increased mean linear intercept (MLI)observed in lung tissue sections. Results of IHC (not shown) indicated increased MUC5Ac and HDAC6 in lung sections. (B) Western blot showing differential expression of MUCSAC, FOXA2, FOXA3, HDAC6, Caveolin 1, PAI-1, p53, AC-TUB and SPDEF in AECs isolated from NL and COPD lungs. (C) Bar graphs showing increased expression of MUC5Ac (measure of mucin), HDAC6, FOXA3, and HDAC6, and decreased FOXA2 mRNA expression in AECs isolated from COPD lungs. These results data reveal increased MUC5Ac and HDAC6, and reduced FOXA2 protein and mRNA expression in AECs of human COPD lungs compared to their basal expressions in NL AECs.

FIG. 12A-12B). Histone deacetylase 6 (HDAC6) affected selective autophagy and regulates COPD-associated cilia dysfunction. (A) Accumulation of LC3-II and expression of Beclin-1, ATG5 and p62 were determined by western blotting for NL and COPD AECs. (B) Bar graphs shows increased expression of LC3, Beclin1 and Atg5 in COPD lungs compare to normal (NL). (C) Immunohistochemical (IHC) staining for MAP-LC3 indicated increased expression in COPD lung tissue.

FIG. 13A-13D. CSP7 mitigates the induction of mucus hypersecretion and cilia shortening in COPD AECs. AECs were isolated from NL and COPD lungs. AECs from COPD lungs were treated with or without CSP7 or CP in vitro for 48h. (A) Western Blot images show increased expression of MUCSAC, HDAC6, PAI-1, p53, Caveolin-1, FOXA3, SPDEF and decreased acetylated tubulin (AC-Tubulin; for cilia length) and FOXA2 expression in AEC lysates of COPD lungs that are reversed with CSP7 treatment. (B) Bar graphs show increased expression of MUCSAC, HDAC6, FOXA3 and Caveolin1 mRNA, and decreased expression of FOXA2 mRNA in COPD AECs analyzed by qPCR; this is reversed by CSP7 treatment. Immunofluorescence staining (not shown) revealed increased co-localization of MUCSAC and HDAC6 in AEC lysates of COPD lungs that are reversed with CSP7 treatment. (C) Immunoblotting performed for LC3, Beclin1, ATG5, p62 in AEC lysates of COPD lungs was reversed with CSP7 treatment (results not shown). AECs from COPD lungs were treated with or without CSP7 or CP in vitro for 6 h. Fluorescence microscopy (results not shown) was performed with acridine orange staining (acidic vesicle). Immunofluorescence staining with acridine orange (not shown) revealed increased co-localization of Ac-Tub/LC3 in AECs exposed to COPD vs. diffused staining in PBS treated controls. CSP7 reversed the co-localization of the AC-Tubulin/LC3. (D) Bar graphs showing the number of ciliated cell and cilia length of in AEC lysates of COPD lungs and indicate reversal with CSP7 treatment.

FIG. 14A-14D TSE induced mucus hypersecretion and cilia dysfunction was reduced by CSP7 (A) Western Blot images showing increased expression of MUCSAC, HDAC6, FOXA3, SPDEF, Beclin-1, LC3 and decreased expression of FOXA2 and AC-Tubulin in AECs lysates from normal human lungs (NL) and cells treated with TS extract (TSE) in vitro for 48 h; this effect was reversed with CSP7 treatment. (B) Bar graph of qPCR data) showing increased MUCSAC, HDAC6, FOXA3, and reduced FOXA2 mRNA expression in AECs isolated from NL treated with TSE and reversal of this expression by CSP7 treatment. (C) Western Blot images show increased expression of LC3, Beclin-1, ATG5 and decreased expression of P62 in AECs lysates from human NL treated with TS extract (TSE) in vitro for 48 h, which is reversed by CSP7 treatment. (D) Bar graphs depicting significant decrease in cilia length and number of ciliated cells in TSE AECs, suggesting mucus cell metaplasia, that are significantly improved after treatment with CSP7. Immunofluorescence staining (not shown) indicated increased co-localization of MUSAC and HDAC6 in AECs exposed to TS extract (TSE) vs diffused staining in PBS treated controls. Treatment of TSE-exposed AECs with CSP7 reversed such co-localization. Further, immunofluorescence staining (not shown) revealed increased co-localization of ACTub/LC3 in AECs exposed to TSE vs diffused staining in PBS treated controls. Treatment of TSE-exposed AECs with CSP7 reversed this co-localization.

FIG. 15A-15D. CSP7 delivered by intraperitoneal (IP) injection or nebulization (neb) mitigated TSE lung injury in mice (A) WT mice (n=10/group) were kept in ambient AIR or TSE for 4 hrs/day 5 days/wk as described. After 16 wks, TSE WT mice were left untreated (None) or exposed to formulated CSP7 (5.8 mg) in 30 ml of PBS containing lactose monohydrate (154 mg) or placebo (Pbo) alone 2 hours daily, 5 days/wk for 4 wks using a NEB tower, or injected IP with 1.5 mg/kg of CSP7 or CP daily 5 days/wk for 4 wks. (A) All mice were subjected to CT and lung volume measurements 20 weeks after TSE. Results showed that systemic (IP) or local(neb) administration of CSP7 reduced lung volume, compliance, Elastance, Resistance. Representative H&E staining (not shown) of tissue sections of 20 weeks TSE WT mice, which was reversed in CSP7 (Neb and IP) treated WT mice. (B) A bar graph shows increased mean linear intercept (MLI) observed in lung tissue sections. (C) Lung parameters of 20 week TSE WT mice, which were reversed in CSP7 (Neb and IP) treated WT mice: Lung volume, elastance, compliance and resistance are shown.

FIG. 16A-16B. CSP7 delivered by nebulization (NEB) or intraperitoneal (IP) injection mitigated TSE lung injury in mice. (A) WT mice (n=10/group) were kept in ambient AIR or TSE for 4 h/d 5 day a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 days/wk for 4 wks using a Neb tower, or were injected IP with 1.5 mg/kg of CSP7 or CP daily 5 d a week for 4 wks. (A&B) show Total lung homogenate analyzed for RNA and protein level for Mucus hypersecretion and a metaplasia marker. IHC (results not shown) revealed increased expression of MUCSAc and HDAC6 in lung sections of 20 wks TSE WT mice, which was reversed by CSP7 (Neb and IP) treatment. Immunofluorescence staining (not shown) indicated increased colocalization of MUCSAC and HDAC6 in lung sections of 20 weeks TSE WT mice, which was reversed by CSP7 treatment (Neb and IP)

FIG. 17. CSP7 delivered by nebulization (NEB) or intraperitoneal (IP) injection decreased acetylated α-tubulin and increased LC3 expression WT mice (n=10/group) were kept in ambient AIR or TSE for 4 h/d 5 days a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or were injected IP with 1.5 mg/kg of CSP7 or CP daily 5 d/week for 4 weeks. IHC (results not shown) revealed increased in expression of Ac-Tub (cilia) and LC3 in lung sections of 20 wk TSE WT mice, which was reversed by CSP7 (Neb and IP) treatment. Tissue staining for Ac-Tub (results not shown) in lung trachea sections of 20 weeks TSE WT mice was reversed by CSP7 treatment. Immunofluorescence (images not shown) using acetylated/α-tubulin (cilia) demonstrated after isolation of MTEC a decrease in number of ciliated cell (Ac-Tub isolated from 20 weeks TSE WT mice, which was reversed in CSP7 treatment (shown in bar graph).

FIG. 18A-B. Human (n=4) tissues from control donors (NL) and from COPD patient lungs (n=4) were treated with PBS or 10 μM CSP or CSP7 ex vivo in dishes for 72 h. (A) Bar graphs showing increased expression of MUCSAC, HDAC6, Caveolin1 and FOXA3 mRNA, and decreased expression of FOXA2 mRNA analyzed by qPCR (B) Western Blot images show increased MUCSAc, HDAC6, SPDEF, and decreased Acetylated Tubulin and FOXA2 level in the COPD lung homogenates, which were reversed by treatment with CSP or CSP7.

FIG. 19A-19B. Role of Caveolin 1 in Mucin Hypersecretion and ciliary disassembly WT mice (n=10/group) were kept in ambient AIR or TSE for 4 h/d 5 days a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d/week for 4 weeks using a Neb tower, or IP injected with 1.5 mg/kg of CSP7 or CP daily 5 d a week for 4 wks. (A) IHC images show increased expression of CAV1 in lung sections of 20 weeks TSE WT mice, which was reversed in CSP7 (Neb and IP) treatment. (B) NL and lungs transduced with Ad-Ev or Ad-CAV were examined in a Western Blot that showed increased MUCSAC, HDAC6, SPDEF, FOXA3, Caveolin1, LC3, Beclin1, ATG5 and decreased FOXA2, AC-Tubulin and p62 expression in TSE-treated AECs. This elevation was greater than that observed in TSE treated AECs transduced with ADD CAV1.

FIG. 20A-20C. Role of p53 and PAI-1 in TSE induced mucin hypersecretion and cilia dysfunction in a mouse model. (A) a bar graph shows increased MUCSAC mRNA expression in TSE-treated AECs, which was absent in TSE treated AECs transduced with Lvp53 shRNA. (B) a Western blot shows increased expression of MUCSAC, HDAC6, SPDEF, and FOXA3, and decreased expression of FOXA, AC-Tub(cilia) expression in TSE-treated AECs, which is absent in MUCSAC, HDAC6, SPDEF and elevation in FOXA2, AC-Tubulin in TSE treated AECs transduced with Lvp53 shRNA. (C) a Western blot shows increased expression of MUCSAC, HDAC6, SPDEF, and FOXA3, and decreased expression of FOXA2 and AC-Tub(cilia) in the lung sections of TSE (20 weeks. WT mice, which was reversed in WT mice kept in ambient AIR, as well as in TSE p53^{-/-} and PAI-1^{-/-} mice. IHC (images not shown) revealed increased expression of MUCSAC in the lung sections of TSE (20 wks) WT mice, which was absent in WT mice kept in ambient AIR, and in TSE p53^{-/-} and PAI-1^{-/-} mice.

FIG. 21A-21E. Mechanism CSP7 attenuation of the effect of mucus hypersecretion and ciliary disassembly. (A) AECs were isolated from NL and COPD lungs. AECs from COPD lungs were treated with or without CSP7 or CP in vitro for 48h. Bar graph shows decreased PP2AC and its reversal by CSP7. (B) Bar graph shows elevation of CIP2A and its reversal by CSP7. (C) Western blot shows that levels of protein PP2AC CIP2A, ERK1/2 and MMP12 were reversed by CSP7. (D) COPD lung tissues with CSP7 ex vivo) have reduced protein phosphatase 2A (PP2A) signaling and which was reversed by CSP7. Serine-threonine phosphatase activity for PP2A was determined for each individual and is represented on the Y axis as pm of phosphate liberated per minute. (E) WT mice (n=10/group) were kept in ambient AIR or TSE for 4h/day, 5 day a week as described. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or were injected IP with 1.5 mg/kg of CSP7 or CP daily 5 d/week for 4 weeks, TSE exposure reduced protein phosphatase 2A (PP2A) signaling and this was reversed by CSP7. Serine-threonine phosphatase activity for PP2A was determined for each individual and is represented on the Y axis as pm phosphate liberated per minute.

### DESCRIPTION OF THE PREFERRED EMBODIMENTS

The present inventors conceived that induction of p53 and downstream PAT-1 augments senescence and apoptosis in A₂Cs, and alveolar injury. Their data reveal a newly recognized contribution of increased IL-17A and PAT-1 to the outcomes of A₂C telomere dysfunction and alveolar damage, and MSAc/mucus hypersecretion by AECs, and airway inflammation during chronic TSE

Intervention by administration of CSP7 and is variants, derivatives, multimers, etc., as described herein acts to block telomere dysfunction in A₂Cs and AECs mucus hypersecretion

Such activity can be examined using primary A₂Cs and AECs isolated from control subjects and patients with COPD, and take advantage of local delivery of CSP7 liquid or DP formulation. Also useful in better understanding the mechanisms involved in the disease process being addressed are lentiviral vectors (Lv) harboring AEC or A₂C specific promoter expressing p53Bp 3′UTR sequences, WT and IL-17A^{-/-}, p53^{-/-} and PAT-1^{-/-}, and p53^{ckO}, PAT-1^{cKO} and Trf2^{cKO} mice lacking their expression in A₂Cs or AECs.

The specificity of CSP7 effects at the molecular level in A₂Cs or AECs can be further confirmed using p53Bp 3′UTR sequences as a decoy that targets p53 binding with endogenous PAT-1 mRNAs without inhibiting p53 expression in mice

CSP7 (a competitor for Cav1-mediated signaling) delivered via airways in liquid or DP formulation, is shown to mitigate A₂Cs telomere dysfunction, senescence/apoptosis, air sac enlargement, and AEC metaplasia/mucus hypersecretion in TSE lung injury.

### Peptides Based on the Cav-1 Sequence

The Caveolin-1 (Cav-1) scaffolding domain or peptide (also referred to as CSD or CSP) interferes with Cav-1 interaction with Src kinases mimics the combined effect of uPA and anti-β1-integrin antibody as discussed in more detail below. Native human Cav-1 has a length of 178 amino acids and a molecular weight of 22 kDa. The amino acid sequence of Cav-1 is shown below (SEQ ID NO:3).

| | |
|---|---|
| 1 | MSGGKYVDSE GHLYTVPIRE QGNIYKPNNK AMADELSEKQ |
| | VYDAHTKEID LVNRDPKHLN |
| 61 | DDVVKIDFED VIAEPEGTHS FDGIWKASFT TFTVTKYWFY |
| | RLLSALFGIP MALIWGIYFA |
| 121 | ILSFLHIWAV VPCIKSFLIE IQCISRVYSI YVHTVCDPLF |
| | EAVGKIFSNV RINLQKEI |

As noted above, CSP is the 20 residue peptide underlined above, and has the sequence GIWKASFTTFTVTKYWFYR (SEQ ID NO:2). The preferred peptide of the present invention, designated CSP7 is the heptapeptide fragment FTTFTVT (SEQ ID NO:1) of CSP and is shown double-underlined within the Cav-1 sequence above. CSP7 has the activities shown in the Examples and Figures, below.

In studies disclosed herein, a control peptide for CSP7, which is termed “CP” is a scrambled peptide with the same amino acid composition as he larger CSP (SEQ ID NO:2), but has a different sequence: WGIDKAFFTTSTVTYKWFRY (SEQ ID NO:5).

Modifications and changes may be made in the structure of CSP7, and to create molecules with similar or otherwise desirable characteristics. Such functional derivatives or biologically active derivatives (which terms are used interchangeably) are encompassed within the present invention.

Preferred functional derivatives are addition variants and peptide oligomers/multimers, and the like.

These may be generated synthetically but also by recombinant production, and tested for biological activity of CSP7. A preferred way to measure the activity of the variant is in a competitive binding assay wherein the ability of the peptide variant to compete with binding of soluble caveolin, such as one that is detectably labeled, with soluble uPAR (“suPAR”).

It is understood that distinct derivatives of CSP7 and longer polypeptides comprising CSP7 may easily be made in accordance with the invention, either by chemical (synthetic) methods or by recombinant means (preferred for longer polypeptides).

Included in within the definition of functional variants of CSP7 are addition which preferably comprise an additional 1-5 amino acids at either terminus or at both termini. In other embodiments (which are intended to be distinct from the peptide multimers discussed below), further additional residues may be added, up to about 20 residues. In the addition variant of CSP7, the additional residues N-terminal to, and/or C-terminal to SEQ ID NO:1 (the core CSP7 peptide) may include some of those in the order in which they occur in the native sequence in Cav-1 (SEQ ID NO:4). However, an addition variant cannot be SEQ ID NO:3. Alternatively, other amino acids can be added at either terminus of SEQ ID NO:1, with the understanding that the addition variant must maintains the biological activity and binding activity of CSP7 (at least 20% of the activity, or preferably greater, as is set forth below).

Preferred substitutions variants of CSP7 is a conservative substitutions in which 1 or 2 residues have been substituted by different residue. For a detailed description of protein chemistry and structure, see Schultz G. E. et al., Principles of Protein Structure, Springer-Verlag, New York, 1979, and Creighton, T. E., Proteins: Structure and Molecular Properties, 2^{nd} ed., W.H. Freeman & Co., San Francisco, 1993, which are hereby incorporated by reference. Conservative substitutions and are defined herein as exchanges within one of the following groups:

Phe may be substituted by a large aromatic residue: Tyr, Trp.

Thr may be substituted by a small aliphatic, nonpolar or slightly polar residues: e.g., Ala, Ser, or Gly.

Val may be substituted by a large aliphatic, nonpolar residues: Met, Leu, Ile, Cys.

Even when it is difficult to predict the exact effect of a substitution in advance of doing so, one skilled in the art will appreciate that the effect can be evaluated by routine screening assays, preferably the biological and biochemical assays described herein. The activity of a cell lysate or purified polypeptide or peptide variant is screened in a suitable screening assay for the desired characteristic.

In addition to the 20 “standard” L-amino acids, D-amino acids or non-standard, modified or unusual amino acids which are well-defined in the art are also contemplated for use in the present invention. These include, for example, in the substitution variant or addition variant, β-alanine (β-Ala) and other w-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homo-arginine (hArg); N-acetyl lysine (AcLys); 2,4-diaminobutyric acid (Dbu); 2,4-diaminobutyric acid (Dab); p-aminophenylalanine (Phe(pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

Other compounds may be designed by rational drug design to function in manner similar to CSP7. The goal of rational drug design is to produce structural analogs of biologically active compounds. By creating such analogs, it is possible to produce drugs that are more active or more stable than the natural molecules (i.e., peptides), lower susceptibility to alterations which affect functions. One approach is to generate a three-dimensional structure of CSP7 for example, by NMR or X-ray crystallography, computer modeling or by a combination. An alternative approach is to replace randomly functional groups in the CSP7 sequence, and determine the effect on function.

Moreover, a biologically active derivative has the activity of CSP7 in an in vitro or in vivo assay of binding or of biological activity, such as assays described herein. Preferably the polypeptide inhibits or prevents apoptosis of LECs induced by BLM in vitro or in vivo with activity at least about 20% of the activity of CSP7, or at least about 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%. The derivative may have 100% or even greater activity than CSP7.

The peptide may be capped at its N and C termini with an acyl (abbreviated “Ac”)- and an amido (abbreviated “Am”) group, respectively, for example acetyl (CH₃CO—) at the N terminus and amido (—NH₂) at the C terminus. A broad range of N-terminal capping functions, preferably in a linkage to the terminal amino group are contemplated

The C-terminal capping function can either be in an amide or ester bond with the terminal carboxyl. Any of a number of capping functions that provide for an amide bond are contemplated.

Capping functions that provide for an ester bond are also contemplated.

Either the N-terminal or the C-terminal capping function, or both, may be of such structure that the capped molecule functions as a prodrug (a pharmacologically inactive derivative of CSP7) that undergoes spontaneous or enzymatic transformation within the body in order to release the active drug and that has improved delivery properties over CSP7 (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules. The presence of a capping group contributes to the stability or in vivo half-life of the peptide.

Chemical Derivatives of CSP7

In addition to capping groups as described above which are considered “chemical derivatives” of CSP7, the preferred chemical derivatives of CSP7 may contain additional chemical moieties not normally a part of a protein or peptide which can be introduced to CSP7 (or to an addition variant of CSP7) by known means to constitute the chemical derivative as defined herein. Covalent modifications of the peptide are included within the scope of this invention. Such derivatized moieties may improve the solubility, absorption, biological half-life, and the like. Moieties capable of mediating such effects are disclosed, for example, Gennaro, A R, Remington: The Science and Practice of Pharmacy, Lippincott Williams & Wilkins Publishers; 21^{st} Ed, 2005 (or latest edition)

Such modifications may be introduced into the molecule by reacting targeted amino acid residues of the peptide with an organic derivatizing agent that is capable of reacting with selected side chains or terminal residues. Another modification is cyclization of the peptide—which is generally accomplished by adding terminal Cys residues which can be bonded via a disulfide bond to generate the cyclic peptide. Alternative, a cross-linkable Lys (K) is added at one terminus and a Glu (E) at the other terminus.

Cysteinyl residues (added, e.g., for cyclizing purposes) most commonly are reacted with α-haloacetates (and corresponding amines) to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitro-phenol, or chloro-7-nitrobenzo-2-oxa-1,3-diazole.

Added lysinyl residues (e.g., for cyclizing) and the amino terminal residue can be derivatized with succinic or other carboxylic acid anhydrides. Derivatization with a cyclic carboxylic anhydride has the effect of reversing the charge of the lysinyl residues. Other suitable reagents for derivatizing amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Derivatization with bifunctional agents is useful for cross-linking the peptide or oligomer or multimer to a water-insoluble support matrix or other macromolecular carrier. Commonly used cross-linking agents include 1,1-bis(diazoacetyl)-2-phenylethane, glutaraldehyde, N-hydroxysuccinimide esters, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Multimeric or Oligomeric CSP7 Peptides

The present invention also includes longer peptides built from repeating units of CSP7 (or a functional derivative thereof) that has the anti-apoptotic and protective activity of CSP7. The preferred peptide unit of such a multimer is FTTFTVT (SEQ ID NO:1). Addition variants of this peptide that may be the “unit” of the multimer preferably include from 1-4 additional amino acids.

A peptide multimer may comprise different combinations of peptide monomers (which may include either or both of SEQ ID NO:1 or addition variants thereof or a chemically derivatized form of the peptide. Such oligomeric or multimeric peptides can be made by chemical synthesis or by recombinant DNA techniques as discussed herein. When produced by chemical synthesis, the oligomers preferably have from 2-5 repeats of a core peptide sequence, and the total number of amino acids in the multimer should not exceed about 160 residues, preferably not more than 100 residues (or their equivalents, when including linkers or spacers).

A preferred synthetic chemical peptide multimer has the formula

P¹ₙ

wherein the core peptide P¹ is SEQ ID NO:1, and wherein n=2-5, and wherein the core peptide alone or in oligo- or multimeric form has the biological activity of CSP7 as disclosed herein in an in vitro or in vivo bioassay of such activity.

In another embodiment, a preferred synthetic chemical peptide multimer has the formula

(P¹-Xₘ)ₙ-P²

P¹ and P² are the core peptides described above, including additional variants, wherein
(a) P¹ and P² may be the same or different; moreover, each occurrence of P¹ in the multimer may be a different peptide (or variant);
(b) X is a spacer which comprises or consists of:
   (i) a short organic chain, preferably C₁-C₅ alkyl, C₁-C₅ alkenyl, C₁-C₅ alkynyl, C₁-C₅ polyether containing up to 4 oxygen atoms, wherein m=0 or 1 and n=1-7; or
   (ii) Gly_{z} wherein, z=1-6, and wherein the core peptide alone or in multimeric form has the biological activity of CSP7 as disclosed herein in an in vitro or in vivo assay of such activity.

When produced recombinantly, a preferred spacer is Gly_{z} as described above, where z=1-6, and the multimers may have as many repeats of the core peptide sequence as the expression system permits, for example from two to about 25 repeats. A preferred recombinantly produced peptide multimer has the formula:

(P¹-Gly_{z})ₙ-P²

wherein:
(a) P¹ and P² are, independently, SEQ ID NO:1 or 3 or an addition variant or derivatized form thereof, wherein P¹ and P² may be the same or different; moreover, each occurrence of P¹ in the multimer may be a different peptide (or variant);
   wherein

n=1-25 and z=0-6; (preferred ranges of n include n=1-5, 1-10, 1-15, or 1-20) and wherein the core peptide alone or in multimeric form has the biological activity of CSP7 as disclosed herein in an in vitro or in vivo bioassay of such activity.

In the present peptide multimers, either P¹ or P² is preferably SEQ ID NO:1. The multimer is optionally capped. It is understood that such multimers may be built from any of the peptides or variants described herein. It is also understood that the peptide multimer should be different from SEQ ID NO:3 (i.e., not native human Cav-1 and should not be a native mammalian Cav-1 homologue).

Peptidomimetics

Also included within the scope of this invention is a peptidomimetic compound which mimics the biological effects of CSP7. A peptidomimetic agent may be an unnatural peptide or a non-peptide agent that recreates the stereospatial properties of the binding elements of CSP7 such that it has the binding activity and biological activity of CSP7. Similar to a biologically active CSP7 peptide, peptide multimer, a peptidomimetic will have a binding face (which interacts with any ligand to which CSP7 binds) and a non-binding face. Again, similar to CSP7, the non-binding face of a peptidomimetic will contain functional groups which can be modified by coupling various therapeutic moieties without modifying the binding face of the peptidomimetic. A preferred embodiment of a peptidomimetic would contain an aniline on the non-binding face of the molecule. The NH₂-group of an aniline has a pKa˜4.5 and could therefore be modified by any NH₂-selective reagent without modifying any NH₂ functional groups on the binding face of the peptidomimetic. Other peptidomimetics may not have any NH₂ functional groups on their binding face and therefore, any NH₂, without regard for pKₐ could be displayed on the non-binding face as a site for conjugation. In addition, other modifiable functional groups, such as —SH and —COOH could be incorporated into the non-binding face of a peptidomimetic as a site of conjugation. A therapeutic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptidic nature.

Peptidomimetic compounds, either agonists, substrates or inhibitors, have been described for a number of bioactive peptides/polypeptides such as opioid peptides, VIP, thrombin, HIV protease, etc. Methods for designing and preparing peptidomimetic compounds are known in the art (Hruby, V J, Biopolymers 33:1073-1082 (1993); Wiley, R A et al., Med. Res. Rev. 13:327-384 (1993); Moore et al., Adv. in Pharmacol 33:91-141 (1995); Giannis et al., Adv. in Drug Res. 29:1-78 (1997). Certain mimetics that mimic secondary structure are described in Johnson et al., In: Biotechnology and Pharmacy, Pezzuto et al., Chapman and Hall (Eds.), NY, 1993. These methods are used to make peptidomimetics that possess at least the binding capacity and specificity of the CSP7 peptide and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the three-dimensional structure of a peptide of the invention either free or bound in complex with a ligand (e.g., soluble uPAR or a fragment thereof). Alternatively, the structure of a peptide of the invention bound to its ligand can be gained by the techniques of nuclear magnetic resonance spectroscopy. Greater knowledge of the stereochemistry of the interaction of the peptide with its ligand or receptor will permit the rational design of such peptidomimetic agents. The structure of a peptide or polypeptide of the invention in the absence of ligand could also provide a scaffold for the design of mimetic molecules.

Deliverable Peptides and Peptide Multimers

One embodiment of the invention comprises a method of introducing the peptide of the invention into animal cells, such as human cells. Compositions useful for this method, referred to as “deliverable” or “cell-deliverable” or “cell-targeted” peptides or polypeptides comprise a biologically active peptide according to the invention, preferably CSP7, or a functional derivative thereof, or a peptide multimer thereof, that has attached thereto or is associated with, a further component which serves as an “internalization sequence” or cellular delivery system. The term “associated with” may include chemically bonded or coupled to, whether by covalent or other bonds or forces, or combined with, as in a mixture. As used herein, “delivery” refers to internalizing a peptide/polypeptide in a cell, Delivery molecules contemplated herein include peptides/polypeptides used by others to effect cellular entry. See for example, Morris et al., Nature Biotechnology, 19:1173-6, 2001). A preferred strategy is as follows: an apoptosis-inhibiting (“biologically active”) peptide of the invention is bonded to or mixed with a specially designed peptide which facilitates its entry into cells, preferably human cells. This delivery system does not require the delivery peptide to be fused or chemically coupled to biologically active peptide or polypeptide (although that is preferred), nor does biologically active peptide or polypeptide have to be denatured prior to the delivery or internalization process. A disadvantage of earlier delivery systems is the requirement for denaturation of the “payload” protein prior to delivery and subsequent intracellular renaturation. These embodiments are based on known approaches for promoting protein translocation into cells.

One type of “delivery” peptide/polypeptide which promotes translocation/internalization includes the HIV-TAT protein (Frankel, A D et al., Cell 55:1189-93 (1998), and the third α helix from the Antennapedia homeodomain (Derossi et al., J. Biol. Chem. 269:10444-50 (1994); Lindgren, M et al., Trends Pharm. Sci. 21:99-103 (2000); Lindgren et al., Bioconjug Chem. Sep-11:619-26 (2000); Maniti O et al., PLoS ONE 5e15819 (2010). The latter peptide, also known as “penetratin” is a 16-amino acid peptide with the wild-type sequence RQIKIWFQNRRMKWKK (SEQ ID NO:6) or two analogues/variants designated W48F (RQIKIFFQNRRMKWKK, SEQ ID NO:7) and W56F (RQIKIWFQNRRMKFKK, SEQ ID NO:8) (Christiaens B et al., Eur J Biochem 2002, 269:2918-2926). Another variant with both of the above mutations is RQIKIFFQNRRMKFKK (SEQ ID NO:9). Transportan, a cell-penetrating peptide is a 27 amino acid-long peptide containing 12 functional amino acids from the N-terminus of the neuropeptide galanin linked by an added Lys residue to the sequence of mastoparan (Pooga, M et al., FASEB J. 12:67-77 (1998)). The sequence of transportan is GWTLNSAGYLLGKINLKALAALAKKIL (SEQ ID NO:10). Analogues of penetratin and transportan are described by Lindgren et al., (Bioconjug Chem. 2000, supra).

Another protein (family) includes VP22, a herpes simplex virus protein that has the remarkable property of intercellular transport and distributes a protein to many surrounding cells (Elliott, G et al., 1997, Cell 88:223-33; O'Hare et al., U.S. Pat. No. 6,017,735). For example, VP22 linked to p53 (Phelan, A. et al., 1998, Nat Biotechnol 16:440-3) or thymidine kinase (Dilber, M S et al., 1999, Gene Ther 6:12-21) facilitating the spread of linked proteins to surrounding cells in vitro. Also useful are VP22 homologues in other herpes viruses, such as the avian Marek's Disease Virus (MDV) protein UL49, that shares homology with HSV-1 VP22 (Koptidesova et al., 1995, Arch Virol. 140:355-62) and has been shown to be capable of intercellular transport after exogenous application (Dorange et al., 2000, J Gen Virol. 81:2219). All these proteins share the property of intercellular spread that provide an approach for enhancing cellular uptake of the peptides, variants, and multimers of this invention.

Also included are “functional derivatives” of the above intercellular spreading or “delivery” “delivery” or “internalization” proteins and peptides such as HIV-TAT or VP22 which include homologous amino acid substitution variants, fragments or chemical derivatives, which terms are herein for the biologically active peptides. A functional derivative retains measurable translocation or intercellular spreading (VP22-like) activity that promotes entry of the desired polypeptide, which promotes the utility of the present biologically active peptide e.g., for therapy. “Functional derivatives” encompass variants (preferably conservative substitution variants) and fragments regardless of whether the terms are used in the conjunctive or the alternative.

Because the above transport proteins are said to work best when conjugated or otherwise bound to the peptide they are transporting, such as CSP7 or a variant or multimer thereof, there are a number of disadvantages to using them. A more effective delivery polypeptide that can be admixed with the biologically active peptide and does not need to be chemically bonded for its action is described in Morris et al., supra, as “Pep-1” which has the amphipathic amino acid sequence KETWWETWWTEWSQPKKKRKV (SEQ ID NO:11). Pep-1 consists of three domains:
(1) a hydrophobic Trp-rich motif containing five Trp residues KETWWETWWTEW (residues 1-12 of SEQ ID NO:11, above). This motif is desirable, or required, for efficient targeting to cell membrane and for entering into hydrophobic interactions with proteins;
(2) a hydrophilic Lys-rich domain KKKRKV (the 6 C-terminal residues of SEQ ID NO:11) which is derived from the nuclear localization sequence of SV40 virus large T antigen, and improves intracellular delivery and peptide solubility; and
(3) a spacer “domain” SQP (3 internal residues of SEQ ID NO:11) which and separate the two active domains above and include a Pro that improves flexibility and integrity of both the hydrophobic and hydrophilic domains.

Accordingly, another embodiment of the invention is a deliverable peptide or polypeptide comprising CSP7 or a functional derivative thereof as described above, and a delivery or translocation-molecule or moiety bound thereto or associated therewith. The delivery molecule may be a peptide or polypeptide, e.g.,
(a) HIV-TAT protein or a translocationally active derivative thereof,
(b) penetratin having the sequence RQIKIWFQNRRMKWKK (SEQ ID NO:8),
(c) a penetratin variant W48F having the sequence RQIKIFFQNRRMKWKK (SEQ ID NO:7)
(d) a penetratin variant W56F having the sequence RQIKIWFQNRRMKFKK, SEQ ID NO:8)
(e) a penetratin variant having the sequence RQIKIWFQNRRMKFKK, SEQ ID NO:9)
(f) transportan having the sequence GWTLNSAGYLLGKINLKALAALAKKIL (SEQ ID NO:10)
(g) herpes simplex virus protein VP22 or a translocationally-active homologue thereof from a different herpes virus such as MDV protein UL49; or
(h) Pep-1, having the sequence KETWWETWWTEWSQPKKKRKV (SEQ ID NO:11).

When a delivery moiety, such as the peptides and proteins discussed above, is conjugated or fused to the biologically active peptide of the invention, it is preferred that the delivery moiety is N-terminal to the biologically active peptide.

In Vitro Testing of Compositions

The compounds of this invention are tested for their biological activity, e.g., anti-apoptotic activity, their ability to affect expression of uPA, uPAR and PAI-1 mRNAs, inhibit apoptosis and senescence of AECs and A₂Cs, etc. using any one of the assays described and/or exemplified herein or others well-known in the art.

In Vivo Testing of Compositions

The ability of a CSP7 variant or multimers to inhibit emphysema, mucus hypersecretion, lung fibrosis in TSE or BLM-treated mice is a preferred test for assessing the functional activity of the compound. Other tests known in the art that measure the same type of activity may also be used.

Pharmaceutical and Therapeutic Compositions and their Administration

The compounds that may be employed in the pharmaceutical compositions of the invention the peptide compounds described above, as well as the pharmaceutically acceptable salts of these compounds. “Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, flowability and solubility of compounds. See, e.g., H. Ansel et al., Pharmaceutical Dosage Forms and Drug Delivery Systems (6^{th} Ed. 1995) at pp. 196 and 1456-1457.

The compounds of the invention, as well as the pharmaceutically acceptable salts thereof, may be incorporated into convenient dosage forms, such as capsules, impregnated wafers, tablets or preferably, injectable preparations. Solid or liquid pharmaceutically acceptable carriers may be employed. “Pharmaceutically acceptable,” such as pharmaceutically acceptable carrier, excipient, etc., means pharmacologically acceptable and substantially non-toxic to the subject to which the particular compound is administered.

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Gennaro, A R, Remington: The Science and Practice of Pharmacy, Lippincott Williams & Wilkins Publishers; 21^{st} Ed, 2005 (or latest edition).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

The present invention may be used in the treatment of any of a number of animal genera and species, and are equally applicable in the practice of human or veterinary medicine. Thus, the pharmaceutical compositions can be used to treat domestic and commercial animals, including birds and more preferably mammals, most preferably humans.

The term “systemic administration” refers to administration of a composition such as the peptides described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as inhalation or instillation in the lung, the preferred route, intraperitoneal, intrathecal, subdural, or to a specific organ. Other examples include intranasal, which is one route that corresponds to instillation in the lungs, intrabronchial, intra-aural or intraocular, etc. The term “local administration” refers to administration of a composition or drug into a limited, or circumscribed, anatomic space, such as subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration. Instillable, injectable or infusible preparations can be prepared in conventional forms, either as solutions or suspensions, solid forms suitable for solution or suspension in liquid prior to injection or infusion, or as emulsions. Though the preferred regional routes of administration are into the lungs, the pharmaceutical composition may be administered systemically or topically or transdermally either separately from, or concurrently with, instillation into the lungs.

Other pharmaceutically acceptable carriers for compositions of the present invention are liposomes, pharmaceutical compositions in which the active polypeptide is contained either dispersed or variously present in corpuscles consisting of aqueous concentric layers adherent to lipidic layers. The active polypeptide is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired.

### Therapeutic Methods

The methods of this invention may be used to treat lung conditions or inflammatory lung diseases such as COPD/emphysema, severe asthma, al anti-trypsin deficiency, cystic fibrosis, bronchiectasis, sarcoidosis, bronchiolitis obliterans, transplant rejection including that resulting from allograft fibrogenesis in a subject in need thereof.

The term “treating” is defined broadly to include, at least the following: inhibit, reduce, ameliorate, prevent, reduce the occurrence or recurrence, including the frequency and/or time to recurrence, or the severity of symptoms of the disease or condition being treated or prevented. This may occur as a result of inhibiting epithelial cell death, inhibiting fibroblast proliferation, any of the other biological or biochemical mechanisms such as telomere shortening that is disclosed herein as being associated with or responsible for the disease being treated.

The CSP7 peptide or peptide derivative or pharmaceutically acceptable salt thereof is preferably administered in the form of a pharmaceutical composition as described above.

Doses of the compound preferably include pharmaceutical dosage units comprising an effective amount of the peptide. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active compound for the treatment of, and sensitivity of, individual subjects

By an effective amount is meant an amount sufficient to achieve a regional concentration or a steady state concentration in vivo which results in a measurable reduction in any relevant parameter of disease.

The amount of peptide or derivative selected, the precise disease or condition, the route of administration, the health and weight of the recipient, the existence of other concurrent treatment, if any, the frequency of treatment, the nature of the effect desired, and the judgment of the skilled practitioner.

A preferred single dose, given once daily for treating a subject, preferably a mammal, more preferably human who his suffering from or susceptible to IPF, COPD or emphysema resulting therefrom is between about 0.2 mg/kg and about 250 mg/kg, preferably between about 10 mg/kg and about 50 mg/kg, for example, via instillation (by inhalation). Such a dose can be administered daily for anywhere from about 3 days to one or more weeks. Chronic administration is also possible, though the dose may need to be adjusted downward as is well-understood in the art. The foregoing ranges are, however, suggestive, as the number of variables in an individual treatment regime is large, and considerable excursions from these preferred values are expected.

For continuous administration, e.g., by a pump system, a total dosage for a time course of about 1-2 weeks is preferably in the range of 1 mg/kg to 1 g/kg, preferably 20-300 mg/kg, more preferably 50-200 mg/kg. After such a continuous dosing regimen, the total concentration of the active compound is preferably in the range of about 0.5 to about 50 μM, preferably about 1 to about 10 μM.

An effective concentration of the active compound for inhibiting or preventing inhibiting apoptosis in vitro is in the range of about 0.5 nM to about 100 nM, more preferably from about 2 nM to about 20 nM. Effective doses and optimal dose ranges may be determined in vitro using the methods described herein.

Treatment of COPD may also include the use of known agents and methods that are helpful in treating or alleviating the symptoms of COPD. These include

(a) Bronchodilators, usually administered using an inhaler to relax the airway smooth muscles, help relieve coughing and shortness of breath and make breathing easier. Both short-acting and long-acting bronchodilators are useful. Short-acting bronchodilators include albuterol (ProAir HFA, Ventolin HFA, others), levalbuterol (Xopenex HFA), and ipratropium (Atrovent). The long-acting bronchodilators include tiotropium (Spiriva), salmeterol (Serevent), formoterol (Foradil, Perforomist), arformoterol (Brovana), indacaterol (Arcapta) and aclidinium (Tudorza).
(b) Inhaled corticosteroids examples of which are Fluticasone (Flovent HFA, Flonase and budesonide (Pulmicort Flexhaler, Uceris, others) reduce airway inflammation and help prevent exacerbations and are thus particularly useful for people with frequent exacerbations of COPD. Some medications combine bronchodilators and inhaled steroids. Salmeterol and fluticasone (Advair) and formoterol and budesonide (Symbicort) are examples.
(c) Oral corticosteroids in short courses are useful for people with moderate or severe acute exacerbation and prevent further worsening of COPD.
(d) Phosphodiesterase-4 inhibitors are a newer type of drug approved for severe COPD and symptoms of chronic bronchitis. One example is roflumilast (Daliresp) which decreases airway inflammation and relaxes the airways.
(e) Theophylline may help improve breathing and prevent exacerbations.
(f) Antibiotics are used to treat respiratory infections, such as acute bronchitis, pneumonia and influenza, which can aggravate COPD symptoms. Azithromycin was shown to prevents exacerbations.
(g) Oxygen therapy if the patient's blood oxygen is too low. Oxygen may be used during activities or while sleeping, or continuously.
(h) Pulmonary rehabilitation programs generally combine education, exercise training, nutrition advice and counseling.

Treatment of cystic fibrosis (CF) may also include the use of known agents and methods that are helpful in treating or alleviating the symptoms of CF. The goals for these treatments include preventing and controlling infections that occur in the lungs, removing and loosening mucus from the lungs, treating and preventing intestinal blockage, providing adequate nutrition. Useful drugs/medications and methods include (a) antibiotics to treat and prevent lung infections (b) anti-inflammatory medications to lessen swelling in the airways; (c) mucus-thinning drugs to help cough up the mucus which can improve lung function; (c) inhaled bronchodilators that can help keep airways open by relaxing muscles around your bronchial tubes; (d) oral pancreatic enzymes to help digestive tract absorb nutrients. CF due to certain gene mutations may benefit from certain newer drugs like ivacaftor (Kalydeco) which improves lung function and weight, and reduces the amount of salt in sweat. For a certain gene mutation Orkambi combines ivacaftor with lumacaftor which may improve lung function and reduce the risk of exacerbations. Chest physical therapy is used to loosening thick mucus in the lungs Mechanical devices including a vibrating vest or a tube or mask can help loosen lung mucus.

Having now generally described the invention, the same will be more readily understood through reference to the following examples which are provided by way of illustration, and are not intended to be limiting of the present invention, unless specified. The examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Examples 1-7

### Treatment

All studies involving mice were performed according to the approved protocols under the guidelines of Institutional Animal Care and Use Committee. C57BL/6 mice of wild type (WT) as well as two knockout strains, PAI-1^{-/-} and uPA^{-/-} on this genetic background (Jackson Laboratory Bar Harbor, Me.) were used.

Cigarette Smoke

To analyze the effect of cigarette smoke, mice were exposed to passive smoke from 40 research cigarettes over a 2-hour period once (2 h) or twice (4 h) daily for 5 days/week for 20 weeks (˜90 mg/m³ total solid particulates) using a mechanical smoking chamber (Teague Enterprises, Davis, Calif.). Control mice were exposed to ambient air. At the 18th week, peptide treatment at a dosage of 30 μg/20 g body weight was initiated and continued on a daily basis for the next 14 days. At the end of the experiment, the mice were euthanized and used for the experiments.

Bleomycin (BLM)

To analyze the effect of BLM, mice were exposed to BLM (40 μg/20 g body weight) once per two week for 16 weeks. Control mice were exposed to normal saline. At 14^{th} week the peptide treatment at a dosage of 30 μg/20 g body weight was initiated and continued on a daily basis for the next 14 days. At the end of the experiment, the mice were euthanized and used for the experiments.

Isolation of AECs From Lungs

AECs were isolated from C57BL/6 mice of wild type (WT) as well as PAI-1^{-/-} and uPA^{-/-} knockouts following the method of Corti et al. (Am J Respir Cell Mol Biol, 1996, 14:309-15) with minor modifications. AECs from human lungs were isolated by a method described by the present inventors' group (Marudamuthu et al., Am J Pathol 2015; 185:55-68). The AECs were plated on plastic culture dishes pre-coated with anti-CD32 and anti-CD45 antibodies for 2 h at 37° C. The non-adherent cells were collected. The purities of AEC cell preparations exceeded 90%, based on lithium carbonate staining for inclusion bodies. The cells were grown in poly-L-lysine coated plates in growth-supplemented AEC culture medium (AEpiCM) (Sciencell, Carlsbad, Calif., USA) at 37° C. in an incubator supplied with 5% CO₂.

Protein Analysis by Western Blotting

Changes in SP-C (cat. no. sc-13979; Santa Cruz Biotechnology, Santa Cruz, Calif.), p53 (cat. no. sc-6243; Santa Cruz), serine 15 phosphorylated p53 (p53^{S15P}, cat. no. 9284; CellSignaling Technology, Beverly, Mass.), lysine 379-acetylated p53 (p53^{Ac}, cat. no. 2570; CellSignaling Technology), caspase-3 (cat. no. ab32351; Abcam, Cambridge, Mass.), cleaved caspase-3 (cat. no. 9661; CellSignaling Technology), PAI-1 (cat. no. ab66705; Abcam) and β-actin (cat. no. 3700; CellSignaling Technology) expression levels were assessed by Western blotting of AEC-lysates using specific antibodies and enhanced chemiluminescence (ECL) (Thermo, Rockford, Ill.) detection as described previously by the present inventor's group (Shetty S et al., Am J Respir Cell Mol Biol 2012; 47:474-83).

Detection of Telomerase Activity

Telomerase activity was detected using a PCR-based telomeric repeat amplification protocol (“TRAP”) method using the TRAPeze Telomerase Detection Kit® (Intergen, Purchase, N.Y., USA). Briefly, the cells were in lysed in CHAPS lysis buffer and quantified by BCA method, and equal quantity of the protein samples was combined with the reaction mix in RNase-free PCR tubes. The PCR amplification was then performed according to the protocol. The final PCR product was loaded onto a 12.5% non-denaturing PAGE gel. Following electrophoresis, the gel was stained with ethidium bromide, and documented using a gel-doc unit (Bio-Rad Laboratories). The relative quantities of telomerase activity for each sample were calculated according to the instructions provided in the kit-protocol.

Measurement of Terminal Restriction Fragment (Telomeric) Length

For determination of telomeric length the TeloTTAGGG Telomere Length Assay Kit® (Roche Diagnostics GmbH) was used. Briefly, genomic DNA was isolated and digested with Hinf1/Rsa. The digested DNA fragments were then separated by electrophoresis on agarose gel followed by Southern blot transfer. The membrane was then hybridized with a telomere specific digoxigenin (DIG)-labelled probe, incubated with anti-DIG alkaline phosphatase, and documented with chemiluminescence detection in gel-doc unit (Bio-Rad Laboratories). Telomeric length was identified by comparing with the pre-labelled molecular weight marker. The relative telomere length was calculated according to the manufacturer's protocol.

Relative telomere length was also analyzed by qPCR analysis of the genomic DNA as described by Callicott R J et al., (Comp Med 2006; 56:17-22) and Cawthon RM (Nucleic Acids Res 2002; 30:e47-e47). The 36B4 gene was served as the control. The primer sequences are provided in the Table 1.

mRNA Quantitation by Real-Time qPCR

Total RNA was isolated from AECs using TRI reagent and reverse transcribed using impromII Reverse transcription Kit® (Promega, Wis.). The levels of the mRNAs were quantitated using an aliquot of reverse transcribed total RNA and gene-specific primers (Table 1) by real-time PCR as described earlier (Shetty et al., supra; Shetty et al., J Biol Chem 2008; 283:19570-80).

Statistical Analysis

The statistical significance of differences between experimental values were analyzed by one-way ANOVA followed by Tukey's post-hoc test using GraphPad Prism 4.0 software (GraphPad Software Inc., San Diego, Calif.).

### Example 2

### Telomere Shortening in IPF and COPD Patients

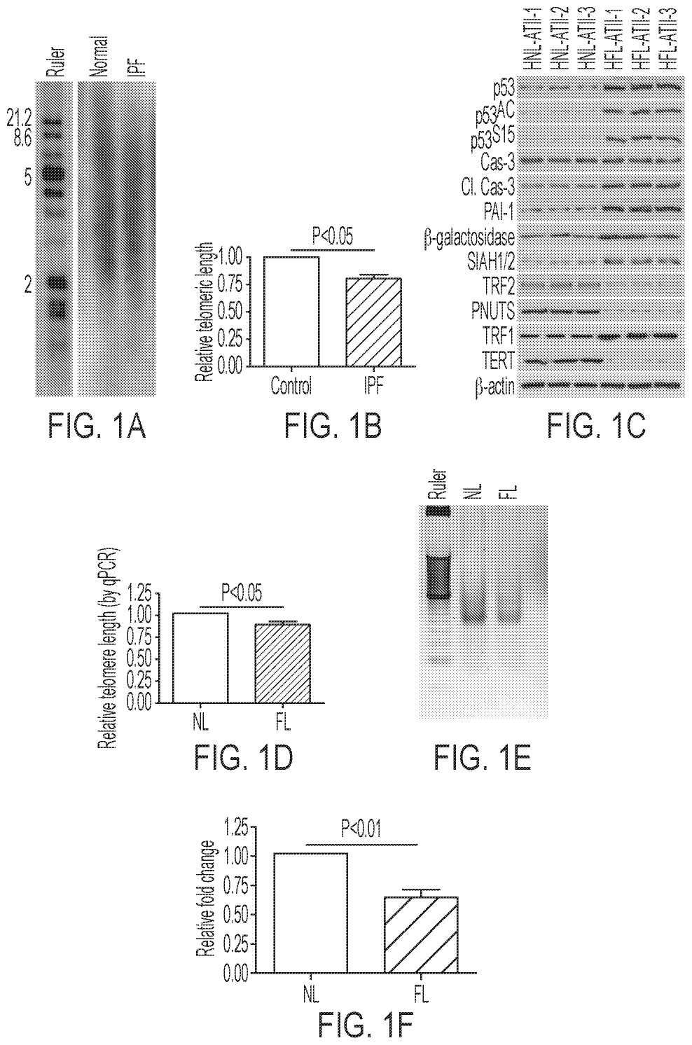

Shortening of the telomere was observed in ATII cells of both IPF and COPD patients. TeloTTAGGG assay showed a significant reduction in ATII-telomere length in IPF patients (FIG. 1), which was substantiated by the qPCR (FIG. 1). The protein expression analyzed by Western blot showed an increase in p53 expression, and p53 activation by acetylation as well as by serine 15 phosphorylation. Activated caspase-3 expression was also increased, implying an increase in apoptosis of ATII cells. Increase in β-galactosidase expression points to the possible increase in a senescence response in the ATII cells.

The expression of SIAH-1, a p53-inducible E3 ubiquitin ligase, known to down regulate the telomere repeat binding factor 2 (TRF2) expression, was also increased.

Downregulation of telomerase reverse transcriptase (TERT) was observed, and was correlated with the TRF2 expression.

Upregulation in the expression of TRF1 was observed; TRF1 is known to suppress the expression of TERT enzyme. The TERT enzyme activity was also significantly downregulated when analyzed by the TRAPeze enzyme assay method. Immunohistochemical analysis has shown that expression of TRF2 and TERT are downregulated, whereas p53 is upregulated in the IPF lung sections.

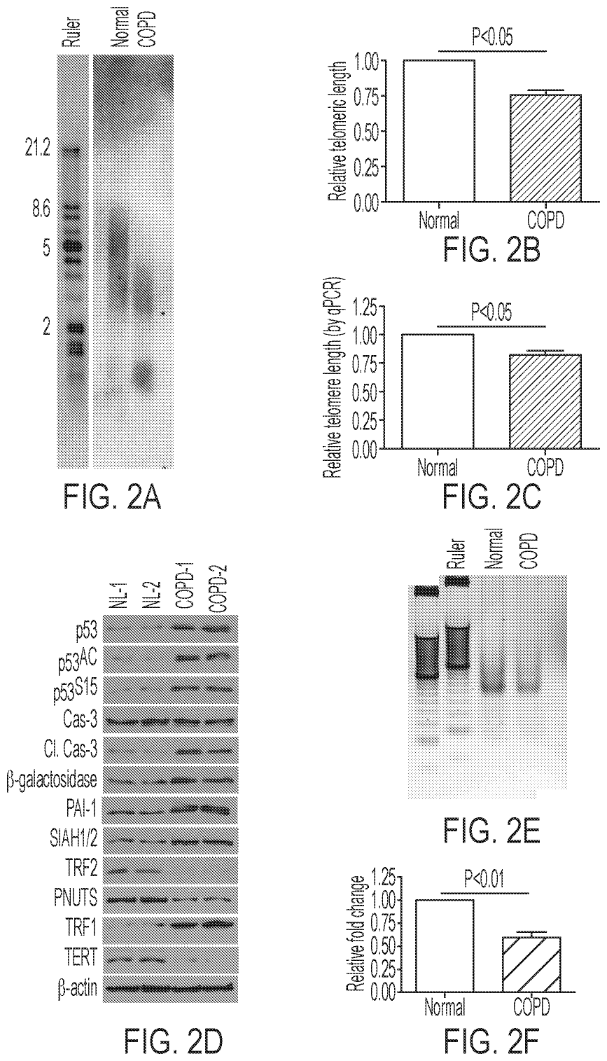

The ATII cells from the COPD patients has also shown a similar pattern of telomere shortening to that of the IPF patients. Telomere length analysis by TeloTAGGG assay and qPCR has shown significant reduction in telomere length (FIG. 2). Protein expression of p53, and p53 activation was also observed with subsequent activation of caspase-3 pointing to the increase in apoptosis in ATII cells. Increase in β-galactosidase expression is pointing to the possible increase in the senescence that ATII cells are undergoing.

Up regulation of the SIAH-1 was observed that might have resulted in the inhibition of TRF2, and subsequent downregulation of the TERT expression observed. In addition, TERT enzyme activity shown significant reduction in ATII cells of COPD patients. Further, the lung sections, when analyzed by immunohistochemistry, has shown downregulation in TRF2 expression, while the p53 and 1 PAI-1 shown increase in their expression.

### Example 3

### Cigarette Smoke and BLM Reduced Telomere in ATII Cells of WT Mice

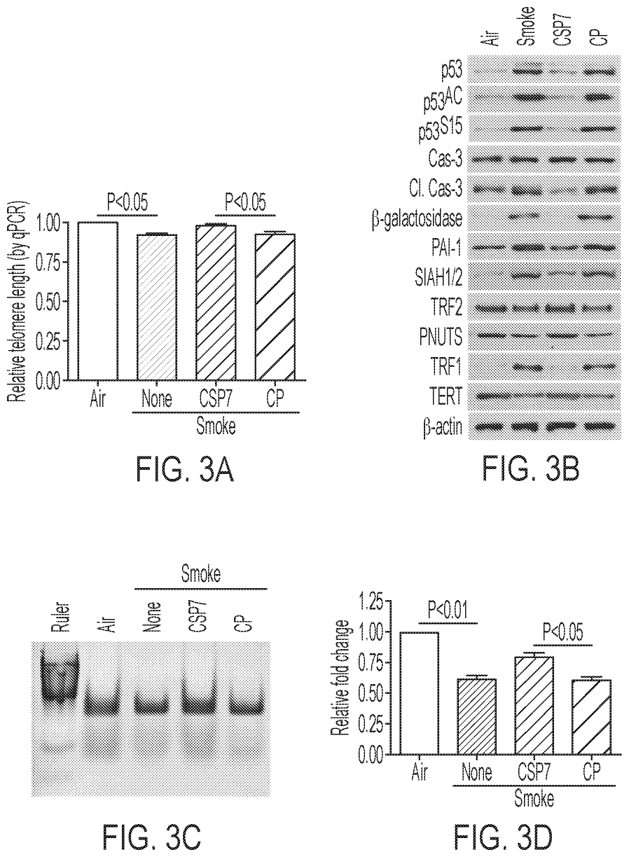

The ATII cells from WT mice treated with 20 week of smoke also showed a significant reduction in telomere length when analyzed by qPCR (FIG. 3), though the extent of the telomere reduction was less severe than that observed in COPD and IPF patients.

Mice treated with the peptide CSP7 showed a significant resistance in telomere shortening when compared with that of the group received the control peptide CP.

Increased protein expression of p53, cleaved caspase-3 and β-galactosidase was observed, indicating progressive ATII cell death.

ATII cells from the CSP7—treated group showed a significant decrease in p53, cleaved caspase-3 and β-galactosidase expression versus the control CP-treated group. Downregulation of SIAH-1 was significantly more in CSP7 group compared to the control CP group.

The CSP7-treated group also showed restoration of TRF2 and TERT expression. The enzyme activity of TERT in CSP7-treated group was significantly higher than that of the control CP group.

Immunohistochemical analysis of lung sections also showed restoration of TRF2 expression with reduction in p53 and PAI-1 expression in the CSP7-treated group.

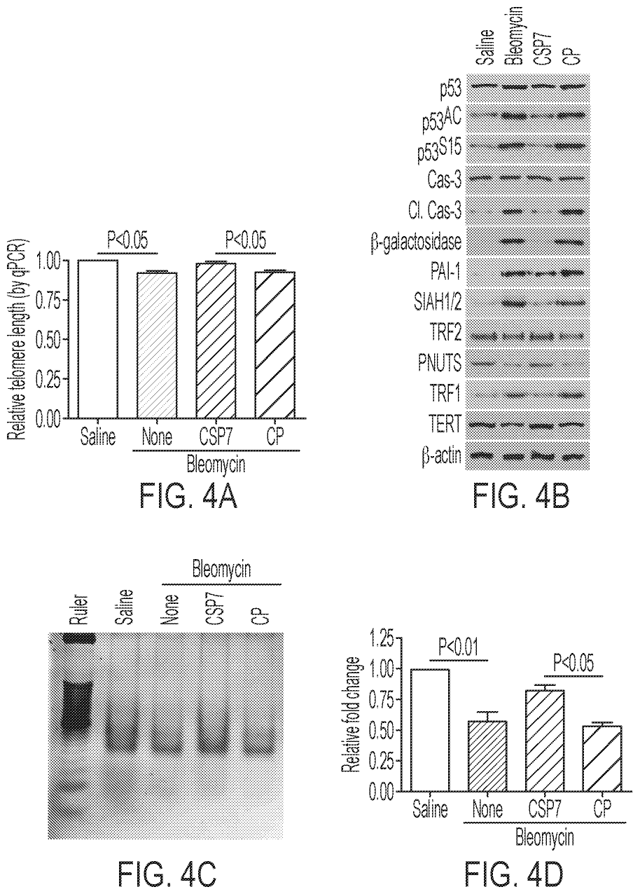

A similar pattern of expression was observed in WT mice whose lungs were damaged by repeated doses of BLM (See FIG. 4).

### Example 4

### Resistance to Telomere Shortening in Mice Lacking miR-34a

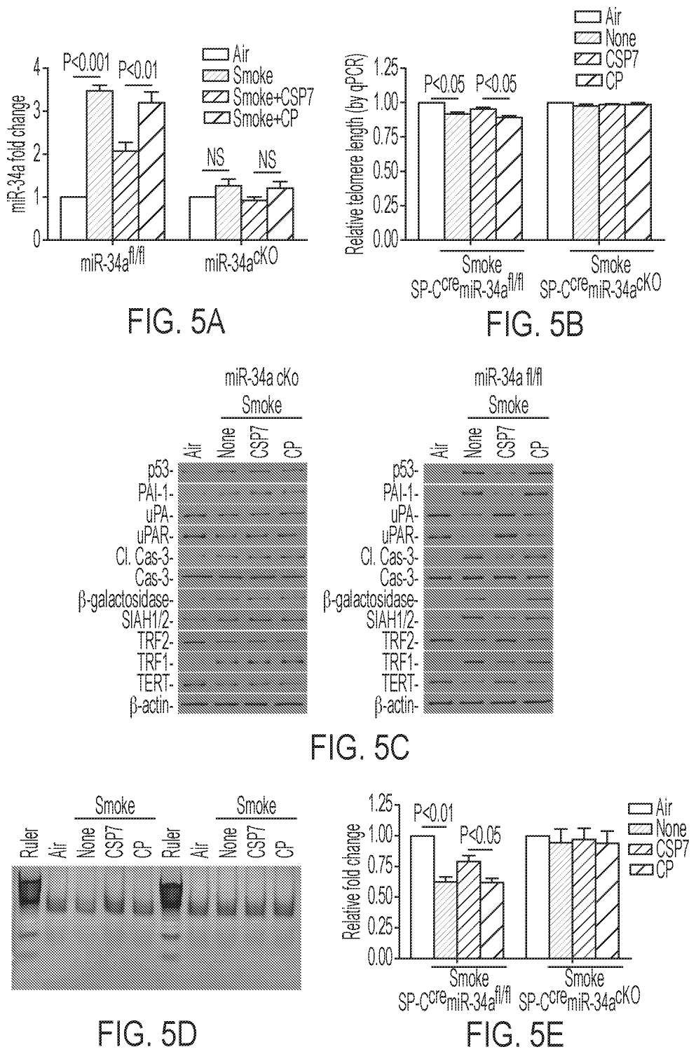

Mice deficient in miR-34a expression in ATII cells, by the CRE gene expression regulated by the SP-C promoter, (miR-34a^{cKO}) shown resistance towards telomere shortening when subjected to 20 week of smoke exposure (FIG. 5). Control mice, which had retained the miR-34a gene (miR-34a^{fl/fl}) were susceptible to telomere shortening by smoke treatment.

CSP7 peptide protected from telomere shortening in miR-34a^{fl/fl} mice similar to that observed in WT mice.

Telomere length analyzed by qPCR, did not show any significant reduction in mice lacking miR-34a expression.

Expression of p53, PAI-1 and active caspase-3 were not upregulated and uPA and uPAR (uPA receptor) expression was not downregulated in miR-34a^{cKO} group in comparison with miR-34a^{fl/fl} group.

Expression of TERT as well as the telomerase enzyme activity was not affected by smoke exposure in miR-34a^{cKO}, whereas significant downregulation in telomerase activity was observed miR-34a^{fl/fl} mice.

CSP7 peptide treatment did not upregulate telomerase activity in miR-34a^{cKO} whereas significant upregulation was observed in miR-34a^{fl/fl} mice receiving CSP7.

### Example 5

### Telomere Shortening in uPA^{-/-} Mice Exposed to Smoke or BLM

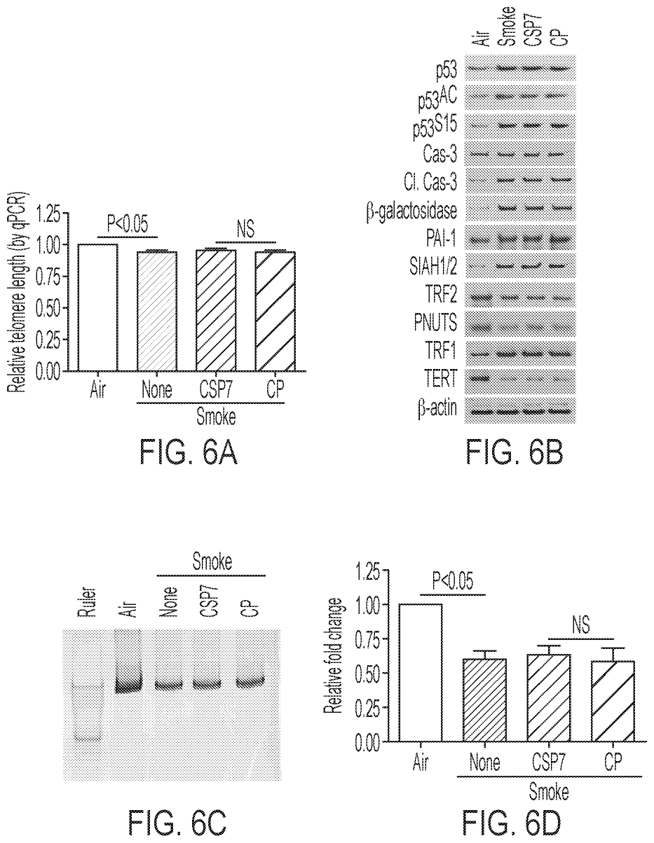

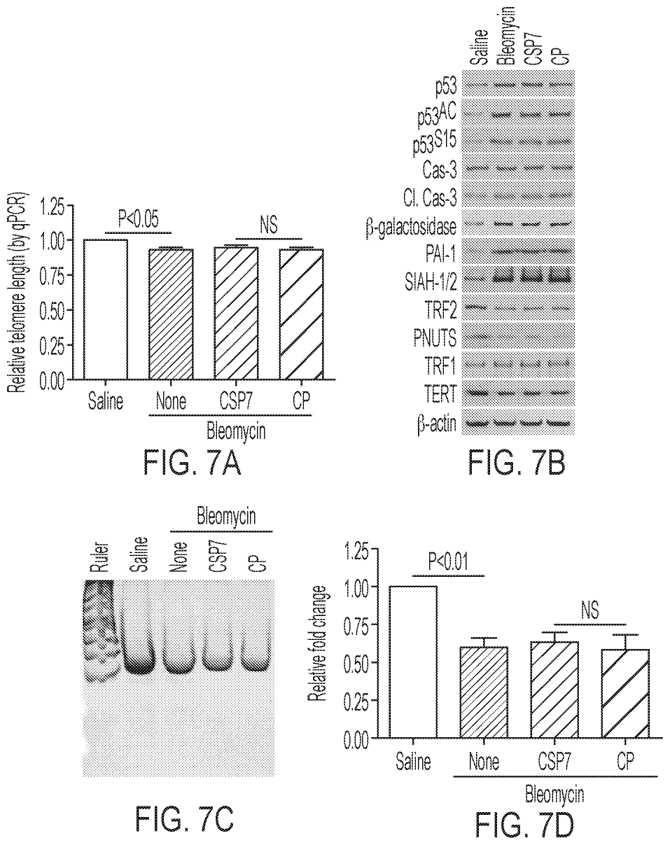

uPA^{-/-} mice exposed to 20 weeks of smoke (FIG. 6) or repeated doses of BLM (FIG. 7) and then treated with the CSP7 did not shown significant difference in telomere shortening compared to the group treated with the control peptide CP.

Treatment with the CSP7 peptide failed to inhibit activation of p53, caspase-3 as well as β-galactosidase when analyzed by Western blotting for protein expression.

There was no significant change in the TRF1, TRF2 and TERT expression between the CSP7- and CP-treated groups.

CSP7 did not restore the telomerase enzyme activity when analyzed by the TRAPeze method.

### Example 6

### Telomere Shortening in PAI-1^{-/-} Mice Exposed to Smoke or BLM

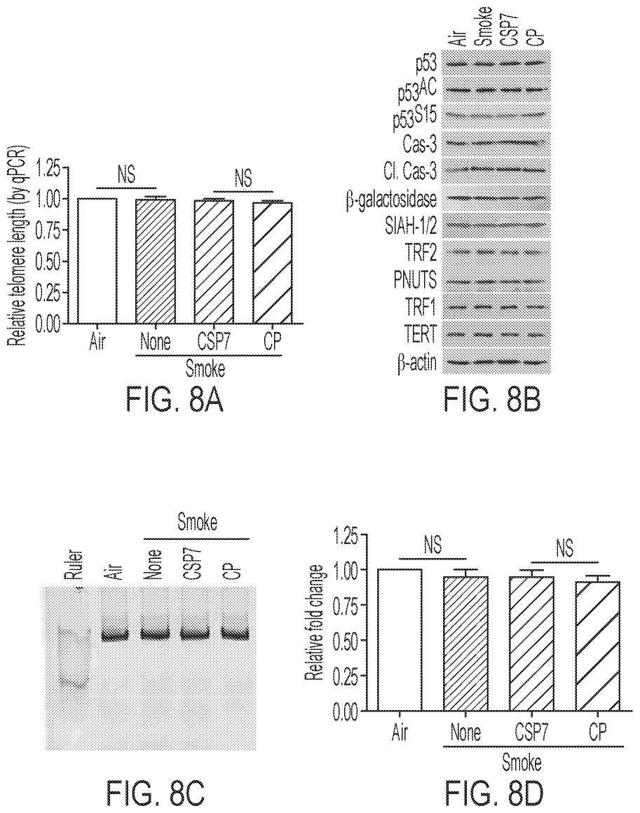

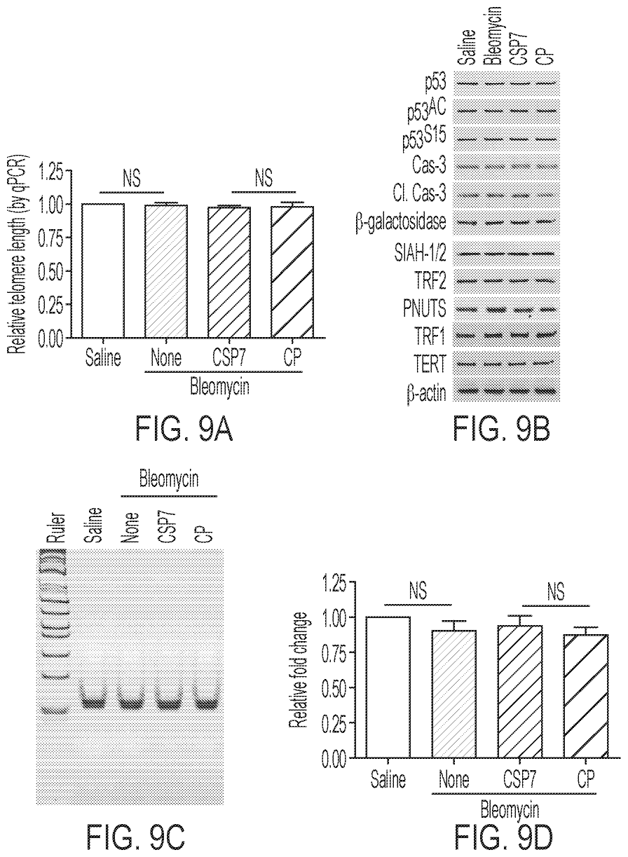

PAI-1^{-/-} mice exposed to 20 weeks of smoke (FIG. 8) or repeated doses of BLM (FIG. 9) were resistant to telomere shortening. There was no significant change in telomere length in the control groups as well as in those received the CSP7 and CP peptides.

Shortening of telomeres analyzed by qPCR did not shown significant changes compared to control groups.

These mice also resisted the activation of p53, caspase-3 and β-galactosidase when analyzed by Western blot for protein expression.

Proteins directly associated with the telomere from the treated group, TRF1, TRF2 and TERT, did not shown any significant changes compared to the control saline/air groups.

Examining telomerase enzyme activity (by TRAPeze assay) also showed that the PAI-1^{-/-} mice were resistant to telomere shortening as there were no significant changes.

### Example 7

### IL-17A-Mediated p53-miR-34a Feed Forward Induction of PAI-1 Contributes to PTS and Bleomycin Lunt Injury

The following summarize the results of these studies.
1. Chronic passive tobacco smoke (PTS) exposure induced accumulation of CD4- and CD8-positive T cells, IL-17A, macrophages and neutrophils in the lungs of WT mice, which was resisted by both p53- and PAI-1-deficient (KO) mice.
2. Treatment of PTS-exposed mice with CSP or CSP7 inhibited pulmonary influx of CD4- and CD8-positive T cells, macrophages and neutrophils.
3. Treatment of WT mice with CSP or CSP7 inhibited PTS-induced accumulation of IL-17A in the lungs.
4. Mice exposed to 20 wks of PTS show increased lung volume indicating emphysema-like condition, which was significantly reduced following treatment of PTS exposed WT mice with either CSP or CSP7.
5. IL-17A treatment induced expression of p53 and PAI-1, and apoptosis in A₂Cs both in vitro and in vivo. Further, the process involved acetylation and serine phosphorylation of p53 proteins in A₂Cs.
6. CSP7 inhibited PTS or IL-17A exposure induced p53 and p53-mediated downstream induction of PAI-1 expression, and apoptosis in A₂Cs both in vivo and in vitro. The process involved inhibition of p53 acetylation through suppression of miR-34a expression and restoration of Sirt1 expression in A₂Cs.
7. IL-17A-deficient mice exposed to PTS resisted induction of p53 or downstream PAI-1 expression or apoptosis in A₂Cs.
8. PTS exposure of IL-17A-deficient mice failed to induce expression of miR-34a or acetylated and total p53 in A₂ECs. IL-17A-deficient mice also resisted PTS exposure induced suppression of baseline Sirt1 expression.
9. Loss of miR-34a expression in A₂Cs prevented PTS induced acetylation of p53 and PAI-1 expression, apoptosis or senescence.
10. Overexpression of miR-34a in A₂Cs alone induced p53 expression and apoptosis.
11. A₂C-specific inhibition of miR-34a expression prevented PTS-induced suppression of Sirt1 expression.

### Discussion of Examples 2-6

(1) The miR-34a and p53 feedback loop is essential for lung inflammation and A₂Cs apoptosis during PTS and IL-17A induced lung injuries.
(2) Elevated miR-34a increased acetylated and total p53, and decreased Sirt1 in WT mice.
(3) Like PTS-induced lung injury, exposure to IL-17A alone upregulated p53, PAI-1 and Cav1 expression, and apoptosis and reduced Sirt1 in WT mice.
(4) IL-17A prevented binding of mdm2 and p53 proteins due to increased acetylation and serine phosphorylation of p53, which results in increased steady state p53 protein level.
(5) IL-17A increased PAI-1 through miR-34a-p53 feed forward induction
(6) CSP7 treatment reduced miR-34a leading to increased
   Sirt1,
   Sirt1-mediated deacetylation of p53 and
   mdm2-mediated degradation of p53.
(7) PTS increased IL-17A and IL-17A receptor, and influx of PMNs and macrophages, and CD4- and CD8-positive T-lymphocytes; these effects were reversed after treatment with CSP7.
(8) PTS and IL-17A failed to induce pulmonary PMN and macrophage accumulation in p53- and PAI-1-deficient mice, suggesting their importance in lung inflammation.
(9) Treatment of mice with IL-17A or Pre-miR-34 caused two-fold increase in total BAL cells. The percentage of PMN in total BAL cells of these treated mouse were 11.27% (IL-17A) and 53.48 (Pre-miR-34a)
(10) Treatment of bronchial epithelial cells with CSP7 inhibited TS-induced MUCSA gene expression indicating that CSP7 is effective against mucus hypersecretion associated with chronic TS exposure.

In conclusion, a mouse model of IL-17A-induced lung injury, as well as comparison of WT and IL-17A-deficient mice exposed to 20 wks of PTS showed an essential role of IL-17A in PTS-induced chronic lung injury, a process that involves miR-34a-p53 feed forward induction and downstream PAI-1 expression.

### Example 7

### CSP7 Inhibits Aging and Age-Associated Diseases by Blocking Telomere Shortening and Mucin Hypersecretion, Inflammation and Acute Lung and Injury and Remodeling

CSP7 inhibits intermediaries affecting telomere shortening/dysfunction in A₂Cs. These effects suggest that CSP7 could be beneficial for treatment of emphysema and aging.

Inhibition of mucin hypersecretion and airway remodeling by CSP7 is useful for treatment of CF, COPD and other diseases associated with excess mucus.

CSP7 would also be used treat wood smoke or other smoke inhalation induced lung injury. CSP7 also be used for bronchopulmonary dysplasia (BPD), hyperoxia induced lung injury, ventilator induced lung injury, silica and other particulate matter induced lung injury and other conditions in which baseline expression of p53 and PAI-1 and lung cell senescence and apoptosis are increased in the lungs.

### Example 8

### Positive Effects of CSP/CSP7 on Lung Dysfunction in a Sepsis Model (Cecal Ligation Puncture, CLP)

This model was described in detail in publication of the present inventor and colleagues, Gao, R et al., Am. J. Physiol. Mol. Physiol, 2015, 308:L847-L853.

Sepsis is initiated and perpetuated by excessive production of inflammatory cytokines and chemokines, resulting in multiple organ failure and death. Lung dysfunction is associated with multiple organ failure during sepsis. Alveolar inflammation, fibrin deposition and alveolar type II cell (A₂C) apoptosis typify acute lung injury (ALI) due to sepsis.

There is no effective treatment to reverse ALI. The present inventors found, in mice with polymicrobial sepsis-induced ALI, that IL-17A induced p53 and apoptosis in A₂Cs, where p53 augmented PAI-1 and inhibited surfactant protein (SP-C) expression. According to the present invention. IL-17A-mediated increases in p53 and PAI-1 in A₂Cs, promote alveolar inflammation and A₂C apoptosis, which are central to sepsis-induced ALI.

While the 20mer peptide CSP was used in the studies noted below, based on results obtained thus far, it is fully expected that CSP7 will have the same effect and is a preferred agent for use in this setting.

1. Induction of p53 and p53-Mediated Downstream Induction of PAI-1 Expression by IL-17A Regulates Sepsis-Induced ALI and ATII Cell Apoptosis.

IL-17A expression increases in the lungs during sepsis-induced ALI, and augments p53 expression in A₂Cs. p53 induces A₂C PAI-1 mRNA and protein expression, with concurrent induction of miR-34a and reciprocal suppression of SP-C expression.

CSP blocks A₂C apoptosis, p53 expression and p53-mediated induction of PAI-1 and ALI via cell surface signaling that involves caveolin-1, β1-integrin and uPAR. Applicant's new data show that sepsis-induced ALI and A₂C apoptosis can be reversed by interrupting this pathway with CSP.

### 2. CSP Inhibits Pulmonary IL-17A Levels, A₂C p53 and PAI-1 Expression, and Apoptosis During Sepsis-Induced ALI.

WT mice were injected IP with vehicle, 1.5 mg/kg of CSP or control peptide (CP) 24 h after CLP injury. Sham-operated mice served as controls. Total RNA isolated from the lungs of these mice 72 h after CLP was quantitated for IL-17A mRNA by real-time PCR.

A₂Cs were isolated from the lungs of these mice 72 h after CLP, and immunoblotted to assess changes in PAI-1, p53 and caspase-3 activation. All were increased by 72h during CLP injury.

Treatment with CSP suppressed sepsis induced IL-17A in the lung tissues with concurrent inhibition of A₂C apoptosis and p53 and PAI-1 expression.

### 3. Increased Interaction Between Caveolin-1 and Protein Phosphatase 2A Catalytic Subunit (PP2A-C) in A₂Cs In Vivo.

Mice were injected IP with CSP or control peptide (CP) 24 h after CLP injury. A₂Cs isolated from these mice 72 h after CLP injury were immunoblotted for caveolin-1 and β-actin. Lysates of A₂Cs from WT mice exposed to the above conditions were immunoprecipitated (IP) for PP2A-C and immunoblotted (IB) for caveolin-1 (Cav-1) to assess their interaction.

Results showed that CLP injury induced caveolin-1 expression in WT mice, and CSP inhibited the A₂C caveolin-1 interaction with PP2A-C.

These results indicate that CSP-mediated changes are associated with inhibition of the caveolin-1 interaction with PP2A-C, an ataxia telangiectasia mutated (ATM) kinase inhibitor that facilitates degradation of p53 by mdm2.¹⁹⁻²¹ This demonstrated a new, intricate link between p53-mediated induction of PAI-1 and apoptosis in A₂C after sepsis-induced ALI.

### 4. CSP Mitigates Lung Inflammation Through Inhibition of PMN Accumulation or Neutrophilia in WT Mice.

Mice treated with or without CSP or CP 24 h after CLP injury were euthanized 72 h later and lung tissues subjected to H&E staining to assess changes in lung inflammation. Lung homogenates and BAL fluids were analyzed for myeloperoxidase (MPO) to access PMN accumulation.

Results showed that CSP significantly inhibited accumulation of PMN, confirming this aspect of CSP-mediated protection against sepsis-induced ALI.

5. CSP Inhibits Sepsis-Induced miR-34a Expression in Mouse Lung A₂Cs In Vivo.

miRNAs, a large group of conserved single stranded non-coding, abundant and short (˜21-25 nt) RNAs which suppress gene expression by targeting mRNAs for degradation or translation repression.

The present results suggest that both miR-34a levels and p53 acetylation are increased in A₂Cs after septic ALI.

CSP treatment of mice after CLP injury inhibited miR-34a by 7-fold in A₂Cs compared to those exposed to CLP or CLP+CP 3 days after injury.

These observations strongly suggest that CSP can reverse IL-17A-mediated induction of A₂C p53, PAI-1 expression, and apoptosis through inhibition of miR-34a.

6. CSP Inhibits CLP-Induced miR-34a Expression in Mouse Lung A₂Cs In Vivo.

Mice were IP injected with CSP, CP or vehicle 24 h after CLP injury. Total RNA isolated from A₂Cs of WT mice with or without CLP, CLP+CSP or CLP+CP 72 h after CLP induced ALI was reverse transcribed and subjected to real time PCR.

miR-34a expression (after normalization for snRNA U6) was significantly reduced in the CSP-treatment group compared to sham-operated controls and peptide controls.

### 7. CSP Induced SP-C Expression by A₂Cs in Mice.

A₂Cs isolated from mice with CLP, CLP+CSP or CLP+CP were immunoblotted for SP-C, thyroid transcription factor-1 (TTF-1) and β-actin. TTF-1 controls transcription of SP-C.

CSP induced SP-C expression by A₂Cs in normal lungs. A₂Cs isolated from mice 72h after IP injection with CSP or CP alone without CLP injury and the lysates were immunoblotted for changes in SP-C expression. Lysates of A₂Cs from uninjured (sham-operated) or CLP mice were also used for comparison.

The results indicated that that CSP-mediated induction of SP-C expression protects A₂Cs from apoptosis during sepsis-induced ALI.

Conclusions: Targeting of p53-mediated induction of PAI-1 and A₂C apoptosis to mitigate sepsis-induced ALI represents a promising novel interventional approach that is supported by the present inventor's recent publications and results described herein. The results further implicate the newly recognized contribution of increased IL-17A with induction of miR-34a, and reciprocal inhibition of SP-C to the outcome of ALI. CSP and CSP7 should reverse septic ALI in patients with sepsis. Thus CSP7 is also used to treat sepsis and streptomycin induced acute lung injury and fibrosis. Since about 40% of patients with ALI develop accelerated lung fibrosis, CSP7 is effective in treating fibrosis caused by Strep infection.

### Example 9

### Materials and Methods for Examples 10-18

### Cell Culture

Primary Airway Epithelial Cells (AECs); Normal, Human (ATCC® PCS-301-010™) and Primary Airway Epithelial Cells; COPD (ATCC® PCS-301-013™) were obtained from the ATCC and cultured in Airway Cell Basal Medium with glutamine, Extract P, HLL Supplement, and AEC Supplement. containing and 1% penicillin-streptomycin. The cells were maintained at 37° C. in a humidified atmosphere at 5% CO₂. All media, supplements, and antibiotics were purchased from ATCC.

### Preparation of TSE for In Vitro Experiments

Research cigarettes 2R4F were purchased from the Tobacco Health Research University of Kentucky (Lexington, Ky.). TSE extracts were prepared by burning research cigarettes in a side arm flask and the smoke generated was bubbled into phosphate-buffered saline at room temperature through an attached peristaltic pump as we described earlier (Bhandary et al PloS One 10: e0123187, 2015) Tiwari et al. Am J Physiol Lung Cell Mol Physiol. 310:L496-506, 2016. An absorbance of 1.0 at 230 nm is considered 100%. TSE extract was filter sterilized by passing it through a 0.2-μm filter.

### Passive TSE Exposure of Mice

Wild-type (WT) and p53- and PAI-1-deficient mice of C57BL/6 background were bred in our facilities or were purchased from Jackson Laboratories. These mice were exposed to passive TSE from 40 research cigarettes over a 2 hour period 5 days/week for 20 weeks (^{˜}90 mg/m3 total solid particulates) by using a mechanical smoking chamber (Teague Enterprises, Davis, Calif.). Control mice were exposed to ambient air. Four weeks after initiation of passive TSE exposure, the mice were administered an intraperitoneal injection of CSP7 or scrambled control peptide (CP) (18.75 mg/kg body wt) once a week for 4 weeks (Marudamuthu et al. Am J Pathol 185: 55-68, 2015); Tiwari et al., supra). Mice were killed, and their lungs were used for further analyses (Bhandary et al., supra).

### Peptide Preparation

CSP7 and CP were dissolved in DMSO and diluted in HBSS for working concentration of 300 μg/2 ml. These peptides were used for the treatment of COPD in vitro and IP injection of mice. For nasal insufflation, the peptides were formulated as follows: 0.579 mg/ml of CSP7/CP was added to 15.456 mg/ml of lactose monohydrate in phosphate buffered saline and the pH was adjusted to 8.4 to give a clear solution. The solution was filtered through a 0.22-micron syringe filter (Bhandary et al. supra; Tiwari et al., supra).

### HE Staining

Three lung tissue sections were randomly Selected from each group. All sections were dewaxed with xylene and hydrated with ethanol. Sections were stained by Hematoxylin and differentiated by hydrochloric acid-ethanol solution. Next, they were counterstained by Eosin, and finally dehydrated by ethanol.

### Immunofluorescence Staining

Airway epithelial cells (AECs) were plated on sterilized coverslips. After treatment, the cells were washed with phosphate buffered saline 3 times, fixed with 4% paraformaldehyde for 20 min, permeabilized with 0.1% Triton X-100 (Biosharp) for 20 min, blocked with 3% bovine serum albumin for 1 h, and then incubated overnight with primary antibody. Subsequently, the cells were stained with FITC-conjugated secondary antibody (Alexis fluor). DAPI was used for nucleus staining (blue). Confocal images of HBE cells were captured with an inverted microscope (Carl Zeiss, Gottingen, Germany) using the Zeiss LSM program.

### Protein Isolation and Western Blot Assay

The cells were lysed with RIPA buffer (Pierce, USA) containing protease inhibitor cocktail (Roche, Germany) and phosphatase inhibitor cocktail (Sigma-Aldrich, USA) on ice for 30 min. After centrifugation at 12,000×g and 4° C. for 20 min, the supernatants were collected. The protein concentrations were determined using the BCA protein assay kit (Pierce, USA). Cell lysates were mixed with 5×SDS-PAGE sample buffer and boiled for 5 min. Thirty micrograms of protein was subjected to 10% SDS-PAGE electrophoresis and transferred to nitrocellulose membranes. The membranes were blocked with 5% milk and then incubated at 4° C. for 16 h with the following diluted primary antibodies (Tiwari et al., supra). The blots were then washed and probed with horseradish peroxidase-conjugated secondary IgG antibodies. The bound antibodies were visualized using SuperSignal™ Maximum Sensitivity Substrate (Thermo Fisher Scientific, USA). For normalization, the membranes were stripped with Restore Western blot stripping buffer and incubated with the following primary antibodies: anti-ERK (1:1000), anti-MUCSAC (1:1000), anti-HDAC6(1:1000), anti-SPDEF(1:1000), anti-FOXA2(1:1000), anti-FOXA3 (1:1000) anti-LC3(1:1000), anti-Beclin1(1:1000), anti-p62(1:1000), anti-p53(1:1000), anti-PAI-1(1:1000) and anti-GAPDH (1:1000).

### Measurement of Pulmonary Function Tests

Pulmonary function tests were performed immediately before CT imaging and before mice were killed, as previously described (DeCologne N et al., Eur Respir J. 35:176-85, 2010). Briefly, mice were anesthetized with a ketamine/xylazine mixture. Anesthetized mice were intubated by inserting a sterile, 20-gauge intravenous cannula through the vocal cords into the trachea. Elastance, compliance, and total lung resistance were then measured (SCIREQ, Tempe, Ariz.). The “snapshot perturbation method” was used to study lung function in the CBB injury model. This method measures total lung resistance, compliance, and elastance of the entire respiratory system. Increased total lung resistance in the CBB model may reflect lung contraction associated with pleural rind formation with concurrent distortion of the airways. The flexiVent was set to a tidal volume of 30 ml/kg at a frequency of 150 breaths/min against 2-3 cm H₂O positive end-expiratory pressure, according to manufacturer's specifications. The mice were maintained under anesthesia using isofluorane throughout the pulmonary function testing.

### CT Scans and Measurements of Lung Volume

After ketamine/xylazine injection, mice were anesthetized further using an isoflurane/O2 mixture to ensure that mice remained deeply anesthetized and to minimize spontaneous breaths. The Explore Locus Micro-CT Scanner (General Electric, GE Healthcare, Wauwatosa, Wis.) was used for CT imaging. CT scans were performed during full inspiration and at a resolution of 93 mm. Lung volumes were calculated from lung renditions collected at full inspiration. Microview software was used to analyze lung volumes and render three-dimensional images (Tucker T A et al., Am J Respir Cell Mol Biol. 50:316-27, 2014).

### PP2A Activity Determination and PP2A Inhibitors

PP2A activity was determined using the Millipore PP2A activity assay (17-313; Millipore) (Nath S, et al., Am J Respir Cell Mol Biol. 59:695-705, 2018 December). Isolation of Mouse Tracheobronchial epithelial cells (MTEC)

Mice were euthanized, after that spray the animal carcasses with 70% ethanol solution to sterilize the yield. With clean surgical scissors and scalpel, skin around the tracheal area was removed, exposing the trachea. The abdomen was opened by cutting along the sternum, and the rib cage was removed exposing tissue up to the end of the trachea. Tracheas were excised and placed into a 50 mL conical tube containing 30 mL Ham's F12 media=antibiotics, on ice. In a sterile lamellar flow hood, tracheal tissue was transferred to a sterile 100 mm Petri dish containing 10 mL Ham's F12 media+antibiotics. Connective tissue was gently dissected with sterile forceps and surgical scissors. Tracheal tissue was placed in a new 100 mm Petri dish containing 10 mL Ham's F12 media+antibiotics to rinse. Tracheas were cut along the vertical axis to expose the lumen. Tracheas were transferred to a 50 mL tube containing 10 mL 0.15% Pronase solution and incubated overnight at 4° C.

DNAse I solution. To 18 mL of Ham's F12 Media+antibiotics, 2 mL of a 10 mg/mL Bovine Serum Albumin (BSA) stock solution was added, along with and 10 mg of crude pancreatic DNAse I. 1 mL aliquots were stored at −20° C. (thawed on ice before use).
Ham's F12 medium with antibiotics with 20% fetal bovine serum (FBS). To 200 mL Ham's F12 basal media (Invitrogen) 50 mL heat inactivated FBS, 2.5 mL of a 100× Penicillin/Streptomycin solution, and 250 μL of a 1000× Fungizone solution were added.
MTEC Basic Medium containing antibiotics. To 475.5 mL DMEM/F12 basic media (Cellgro) 7.5 mL 1 M HEPES, 10 mL of 200 mM glutamine, 2 mL of a 7.5% NaHCO₃, 5 mL of a 100× penicillin/streptomycin, 500 μL of 1000× Fungizone were added. For MTEC medium/10% FBS. 5 mL heat inactivated FBS were added to 45 mL of MTEC basic medium+antibiotics, 10 mL Ham's F12 media containing 20% FBS and antibiotics were added to the tube and rocked 12 times.

### Trachea Preparation

Tracheas were removed from the Pronase solution, setting aside this solution on ice and transferred a conical tube containing Ham's F12; the tube was inverted 12 times and this process repeated twice. Pronase solution was combined with the three supernatants, and remaining tissue was discarded. Tubes were centrifuged at 1400 rpm for 10 min at 4° C., and supernatant discarded. The pellet was gently resuspended in 1 mL DNAse solution (100-200 μL/trachea) and incubated for 5 min on ice and then centrifuged at 1400 rpm for 5 min at 4° C., and the supernatant discarded. The cell pellet was resuspended in 8 mL MTEC medium with 10% FBS. Cell suspensions were plated and incubated at 37° C. in an atmosphere of 95% air, 5% CO₂ for 5 hrs. Cell suspension were collected from plates and the plates rinsed twice with 4 mL MTEC +10% FBS. Cell suspension and washes were pooled in a 50 mL conical centrifuge tube. 1 mL was set aside for cytospin and cell counting. Tubes were centrifuged in a tabletop centrifuge for 5 min at 5,000 rpm. 500 μL were removed and the pellet resuspended in remaining supernatant. 100 μL was taken for viable cell counting. 4 aliquots of 100 μL were set aside for cytospin analysis. The remaining 15 mL cell suspension were centrifuged at 1400 rpm, at 4° C. for 10 min (Lam H C, et al., J Vis Exp 48:2513, 2011).

Treatment of Human Lung Tissues with CSP7

Lung tissues from control subjects and patients with COPD were treated with or without CSP7 for 72 h ex vivo or in vitro. Lung homogenates, were analyzed for immunoblot and Real time PCR.

### Statistical Analysis

All results are representative of at least five independent experiments which were quantified and plotted as the mean±standard deviation. Student's t-test was used for evaluating statistical significance of differences between experimental groups. Further, non-parametric tests for analysis amongst groups were also done using one-way ANOVA Kruskal-Wallis test, with Dunn's multiple group comparison tests as appropriate. Statistical analyses were done using the SPSS Statistics 20 (IBM SPSS software, version: 20.0, Chicago, Ill., USA) and GraphPad Prism 5 (GraphPad Software, Inc., San Diego, Calif., USA). The P value was defined as follows: not significant (ns): P>0.05. P values of *P<0.05; **P<0.01; ***P<0.001 and ****P<0.0001 were considered statistically significant.

### Example 10

### Airway Epithelial Cells (AECs) from Subjects with COPD Shows Differential Expression of MUCSAC, FOXA2, FOXA3, HDAC6, and SPDEF

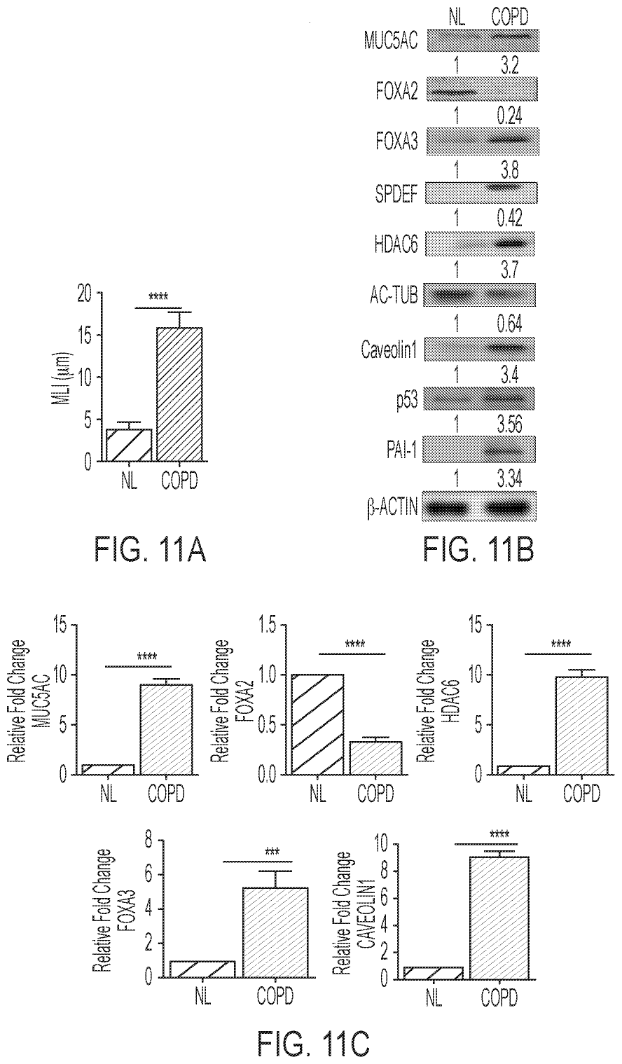

The main problem in emphysema is that the walls of the air sacs are destroyed. The inner walls of the sacs weaken and burst, creating one large space for holding air instead of many small ones. Representative H&E staining of tissue sections of Normal (NL) and COPD and alongside bar graph showing increased mean linear intercept (MLI) observed in lung tissue sections. The mean linear intercept (chord) length (Lm) is a useful parameter of peripheral lung structure as it describes the mean free distance in the air spaces (FIG. 11A Patients with GOLD 4 COPD had an increase in mean linear intercept compared with Normal (NL). MUCSAC as a deleterious and dispensable glycoprotein component of airway mucus. Consistent with prior studies of airway mucin gene expression in humans. In an attempt, we examined whether on MUCSAC, a secreted-polymeric mucin, as it is highly expressed by airway surface mucus producing cells in COPD patients. We found upsurge in MUCSAC and HDAC6 expression in COPD lung as compare to NL. Mucous cell metaplasia is associated with decreased expression of the transcription factor FOXA2 and increased expression of the related transcription factor FOXA3 in COPD Patients.

Nuclear FOXA2 protein expression in airway epithelial cells was reduced during mucous metaplasia. These results indicate that the FOXA2 transgene was expressed in airway epithelial cells and that transgene expression persisted after allergen challenge.

FOXA3 affects mucus production, which might be involved in other aspects of allergic airway disease. Intense expression of FOXA3 was detected in airway goblet cells in tissue from patients with COPD in immunoblot. Histological analysis of COPD patient lung sections showed increased MUCSAC staining as compare to NL.

Immunoblot and Real time PCR were performed to investigate the expression of mucin hypersecretion related genes. The results revealed that decrease of FOXA2 and acetylated a Tubulin(Ac-Tub) levels and increased expression of MUCSAC, HDAC6, SPDEF, and FOXA3 in AECs isolated from human COPD patients compared to their basal expressions in NL AECs (FIGS. 11B and C). Also observed were elevation in Caveolin, PAI-1, p53 expression in COPD AECs as compare to NL (FIG. 11B).

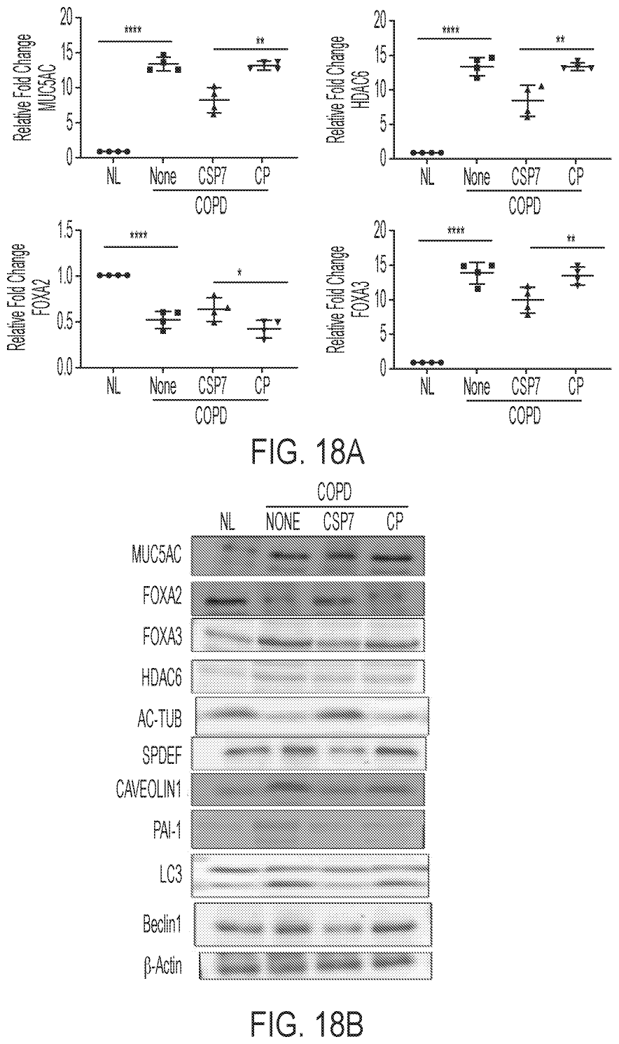

Human NL (n=4) tissues from control donors and COPD lung (n=4) tissues were treated with PBS or 10 μM CSP or CSP7 ex vivo in dishes for 72h. Bar graphs showing increased expression of MUCSAC, HDAC6, Caveolin1 and FOXA3 mRNA, and decreased expression of FOXA2 mRNA was analysed by QPCR (FIG. 18A). Simultaneously, western Blot images shows increased MUC5Ac, HDAC6, SPDEF, and decrease in Acetylated Tubulin and FOXA2 level in the COPD lung homogenates being reverse by treatment with CSP or CSP7 (FIG. 18B).

### Example 11

### Histone Deacetylase 6 (HDAC6) Interceded Selective Autophagy Regulates COPD-Associated Ciliary Dysfunction

Autophagy refers to a dynamic process by which cytoplasmic organelles and proteins are sequestered into autophagosomes that subsequently fuse with lysosomes, leading to the degradation of cargo by lysosomal hydrolases (Mizushima N et al., Cell. 147:728-41, 2011; Yang Z et al., Cell 132:27-42, 2008)

The roles of HDAC6 in motile cilia of the airways, in cellular responses to TSE exposure, and in COPD pathogenesis have not been clarified. Therefore the expression of HDAC6 in lung tissue obtained from COPD patients was assessed. HDAC6 expression was upregulated in lung tissue of COPD patients; increased HDAC6-positive staining was detected in airway epithelia of COPD patients relative to control (NL) subjects. HDAC6 has been shown to regulate primary cilia resorption in response to extracellular stress (Prodromou et al., J Cell Sci. 125(pt 18):4297-4305, 2012) as well as the autophagic pathway through autophagosome-lysosome fusion (Lee et al., EMBO J. 29:969-80, 2010). Moreover, ciliophagy, an HDAC6-dependent autophagic pathway, represents what the inventors consider a novel pathway that is critical to cilia homeostasis in response to TSE exposure. Immunoblots were performed to check the expression of Cilia (acetylated a-tubulin) and diminution expression of acetylated a-tubulin in COPD tissue as compared to NL was found.

In initial experiment, a lysosomotropic agent, acridine orange, was used to detect acidic vesicles. Results indicated that isolated AECs from COPD showed increased late autophagic vacuoles, as evidenced by an increase in fluorescence intensity.

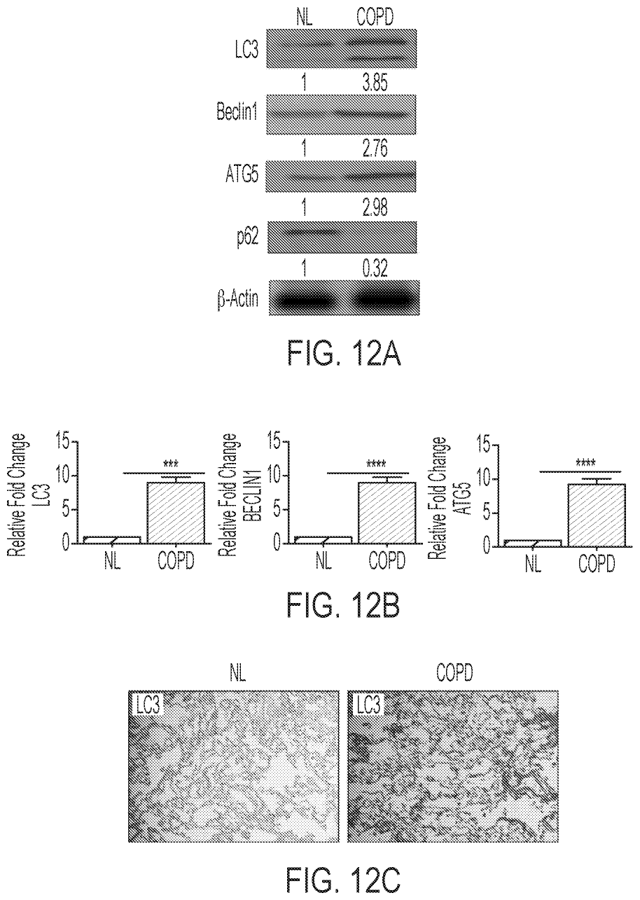

Immunoblot and Real time PCR were performed to examine at both mRNA and protein levels. Increased expression of LC3, Beclin1 and Atg5 were found in COPD lungs compare to normal (NL) (FIGS. 12B&C).

Elevated levels of autophagy protein in COPD lung as compare to NL was observed. Histological analysis for MAP-LC3 showed increased expression in COPD lung tissue. The ratios of LC3B-II/I level, as well as the expression of Atg5 and beclin1, were increased in lung tissue from humans with COPD.

As a marker of autophagic flux, p62 is involved in the degradation of unfolded or misfolded proteins in cells, and the content of insoluble p62 is an indicator of autophagy activation Hua F et al., Nat Commun. 6:7951, 2015).

The amount of insoluble p62, but not soluble, p62 was significantly decreased in lung tissue in COPD as compare to NL, suggesting that it activates autophagy in lung tissue of COPD patients. Interestingly, inconsistent with the increment of LC3B-II level and reduction of insoluble p62 amount, beclin1 levels in COPD airway epithelial cells indicate that COPD-induced autophagy also occurs in AECs (FIG. 12A).

### Example 12

### CSP7 Mitigates the Induction of Mucus Hypersecretion and Cilia Shortening in COPD AECs

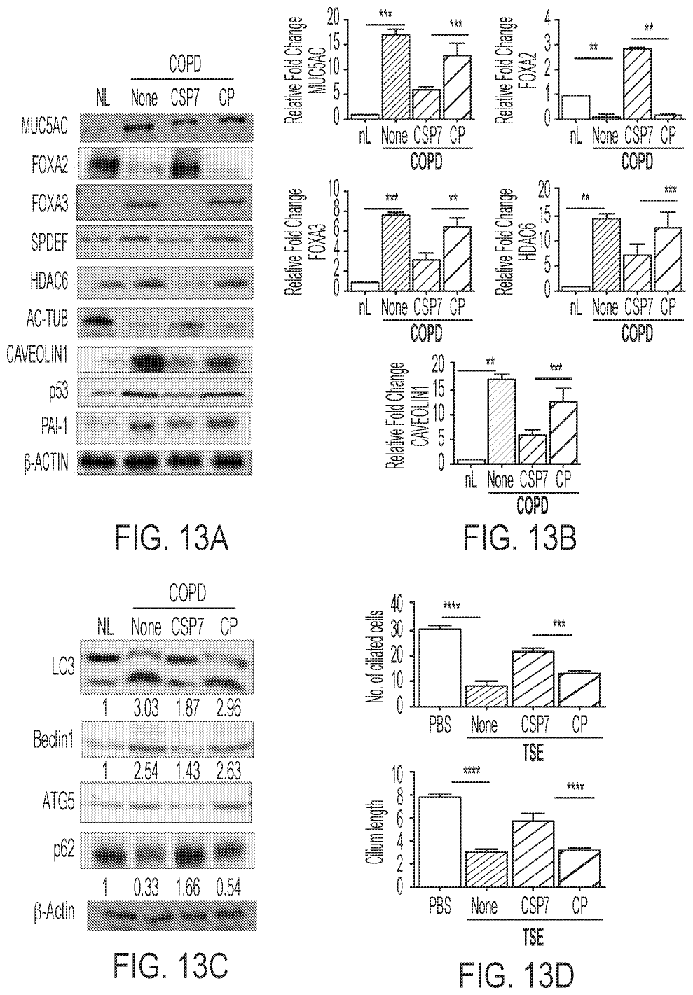

AECs were isolated from NL and COPD lungs. AECs from COPD lungs were treated with or without CSP7 or CP in vitro for 48h. Western Blot images show increased expression of MUCSAC, HDAC6, PAI-1, p53, Caveolin-1, SPDEF and decreased FOXA2, Ac-Tub (for cilia length) in AEC lysates from COPD lungs, and that these are reversed with CSP7 treatment (FIG. 13A). Quantitative PCR showing increased expression of MUC5Ac, HDAC6, and FOXA3 mRNA, and decreased expression of FOXA2 mRNA in COPD AECs, all of which were reversed by CSP7 treatment (FIG. 13B). The lysosomotropic agent, acridine orange was used to detect acidic vesicles; AECs isolated from COPD lungs shows increased late autophagic vacuoles, as evidenced by an increase in fluorescence intensity. This was reversed by CSP7. Further, changes in the expression of endogenous LC3-II in AECs were examined. Rapid accumulation of the LC3-II form (corresponding to characteristic lipidation of this protein during autophagosome formation) was observed in COPD, and was reverse with CSP7. Besides, immunoblots were used to analyze the expression of other autophagic proteins, including Beclin-1 and ATG5. Their elevated expression in COPD was mitigate by CSP7(FIG. 13C). Moreover, the expression of p62 was significantly elevated with CSP7 treatment in COPD AECs. Interestingly, immunofluorescence staining revealed increased co-localization of MUCSAC/HDAC6 and Ac-Tub/LC3 in COPD AECs; this co-localization was reversed by CSP7 (FIG. 13D).

### Example 13

### TSE Induces Mucus Hypersecretion and Cilia Dysfunction which is Reduced by CSP7

The combination of hypersecretion and ciliary impairment leads to disruption of mucociliary interaction, and, hence, the accumulation of secretions in the lower airways. Cigarette smoke appears to play a critical role in the pathogenesis of COPD associated mucociliary dysfunction. While the excessive lower airway secretions may have only minor effects on the natural course of airflow obstruction, they could transiently compromise airway function during acute exacerbations. Furthermore, western blot images showed increased expression of MUCSAC, HDAC6, SPDEF, FOXA3 and decreased expression of FOXA2 and Ac-Tub in AECs lysates from human NL AECs treated with TS extract (TSE) in vitro for 48 h, which was reversed with CSP7 treatment

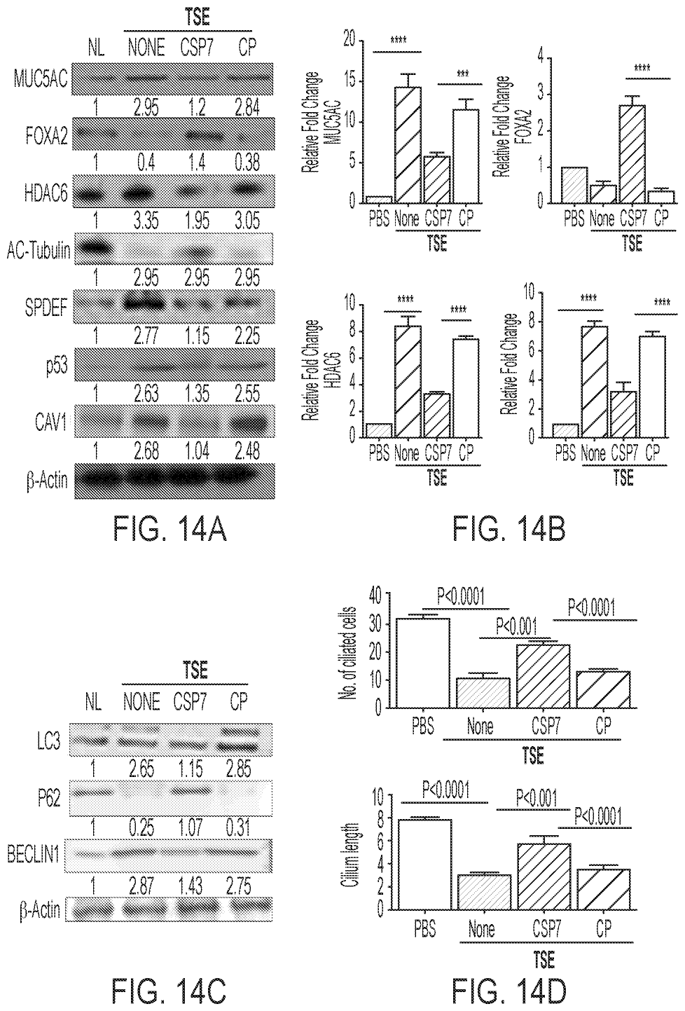

(FIG. 14A). Bar graphs (QPCR data) showed increased MUCSAC, HDAC6, FOXA3 and SPDEF and reduced FOXA2 mRNA expression in AECs isolated from NL treated with TSE; this increased expression was reversed by CSP7 treatment (FIG. 14B). Western Blots for Autophagy protein markers by TSE and was reversed by CSP7 (FIG. 14C).

Moreover, Immunofluorescence staining revealed increased co-localization of MUCSAC/HDAC6 and AC-Tub/LC3 in AECs exposed to TS vs diffused staining in PBS treated controls. TSE-treated AECs exposed to CSP7 showed reversal of the co-localization of the MUCSAC/HDAC6 and AC-Tubulin/LC3. Bar graphs depicting significant decrease in cilia length and number of ciliated cells in TSE AECs show that this was significantly improved after treatment with CSP7 (FIG. 14D).

### Example 14

### CSP7 Delivered by Nebulization (NEB) or Intraperitoneal (IP) Injection Mitigates TSE Lung Injury in Mice

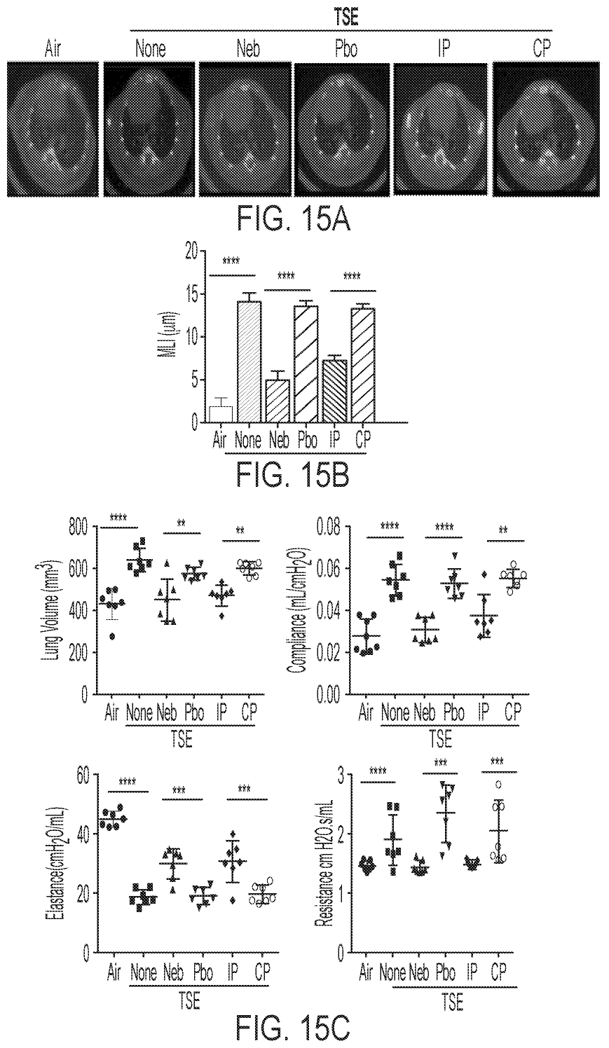

Wild type (WT) mice of the C57BL/6J strain (n=10/group) were kept in ambient air or were TSE for 4 hour/day, 5 days a week as described. After 16 weeks, TSE WT mice were left untreated (“None”) or given formulated CSP7 (5.8 mg) in 30 ml of PBS containing lactose monohydrate (154 mg) or placebo (Pbo) alone 2 h daily 5 days a week for 4 weeks using a Neb tower, or IP injection of 1.5 mg/kg of CSP7 or CP daily 5 days a week for 4 weeks. All mice were subjected to CT and lung volume measurements 20 weeks after TSE exposure (FIG. 15A). Results showed that systemic (IP) or local (Neb) administration of CSP7 reduced lung volume, compliance, elastance and resistance. Besides, representative H&E staining of tissue sections of 20 weeks TSE WT mice, which was reversed in CSP7 (NEB and IP) treated WT mice and bar graphs showing increased mean linear intercept (MLI) observed in lung tissue sections (FIG. 15B-C). Simultaneously, lung parameter of 20 weeks TSE WT mice, like Lung volume, Elastance, compliance and resistance show a trend of reversal with CSP7 (Neb and IP) treatment.

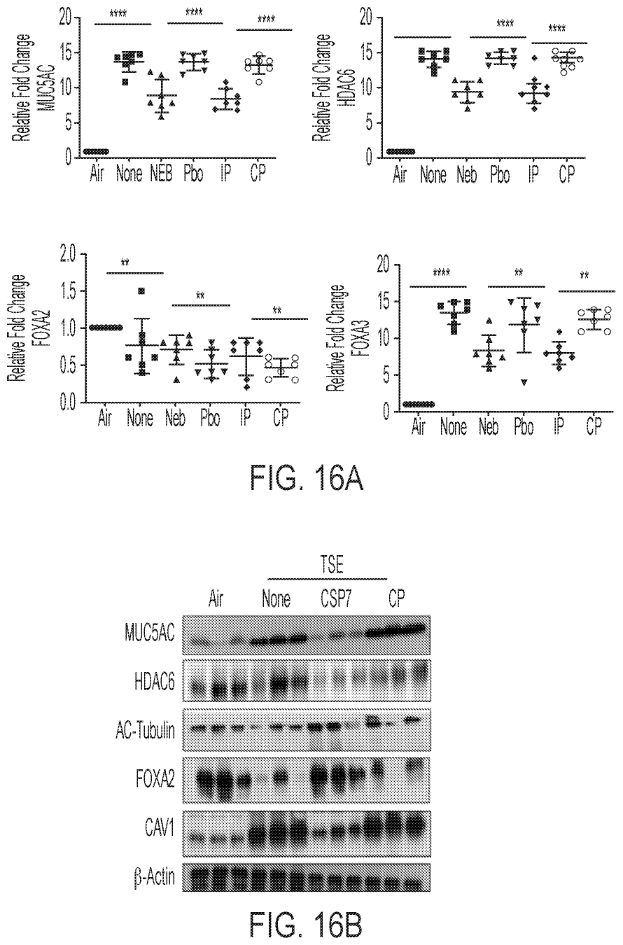

Moreover, CSP7 delivered by intraperitoneal (IP) injection or nebulization (neb) alleviated TSE MUCSAC and HDAC6 expression. Total lung homogenates were analysed for RNA and protein level for Mucus hypersecretion and autophagy marker (FIGS. 16A&B). Histological analysis of lung sections also showed increased expression of MUCSAC and HDAC6 in lung sections of 20 week TSE WT mice, which was reversed by CSP7 (Neb and IP) treated WT mice.

The beneficial effects of locally and systemically delivered CSP7 against TSE induced lung injury provide strong rationale. Additionally, immunocytochemical (ICC) staining revealed increased co-localization of MUCSAC/HDAC6 in lung sections of 20 week TSE WT mice, which was reversed in CSP7 (Neb and IP) treated WT mice.

### Example 15

### CSP7 Delivered by Nebulization (NEB) or Intraperitoneal Injection ((IP)) Decreased Acetylated a-Tubulin and Increased LC3 Expression

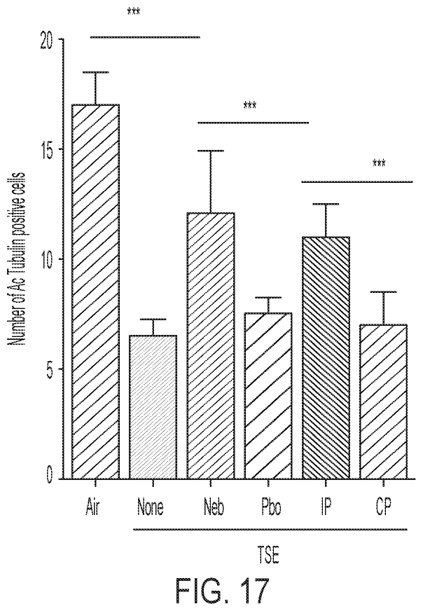

WT mice (n=10/group) were kept in ambient AIR or TSE for 4 hour/day 5 days a week as described. After 16 weeks, TSE WT mice were left untreated (None) or exposed to formulated CSP7 (as above, by NEB or IP injection) or placebo (PBO) alone. Immunohistochemistry (IHC) images showed decreased expression of Ac-Tubulin and increased LC3 expression in lung sections of 20 weeks TSE WT mice, which is was reversed in CSP7 (Neb and IP) treated WT mice (FIG. 17). CSP7 delivered by IP injection or nebulization (NEB) mitigated TSE lung injury in mice. Staining for Ac-Tub in lung trachea sections of 20 weeks TSE WT mice was reversed in CSP7 treated WT mice.

Studies were done to better understand the mechanisms by which TSE exposure disrupts the function of ciliated epithelial cells of the respiratory tract and their impact on airway function. Mouse tracheal bronchial epithelial cells (MTEC) were isolated from 20 weeks TSE WT mice as well as those treated with CSP7 or CP. Interesting, immunofluorescence imaging using acetylated a-tubulin (cilia) after isolation of MTEC, demonstrated decreases in the number of ciliated cell (Ac-Tub isolated from 20 weeks TSE WT mice) and its reversal in CSP7 treated WT mice (Lam H C et al. J Clin Invest 123: 5212-30, 2013).

### Example 16

### Role of Caveolin 1 in Mucin Hypersecretion and Ciliary Disassembly

Caveolin-1, a component protein in the cell membrane, reportedly regulates airway inflammation and lung injury (Yu, Q. et al., Int J Mol Med. 35:1435-42, 2015)

A bar graph shows increased Caveolin-1 mRNA expression in COPD as compare to NL. A goal of this study was to determine whether Caveolin-1 modulates mucin hyperproduction induced by TSE. AECs were isolated from NL and COPD lungs. The cells from COPD lungs were treated with or without CSP7 or CP in vitro for 48h. Bar graphs show increased expression of Caveolin1 mRNA, COPD AECs analysed by QPCR, which was reversed by CSP7 treatment.

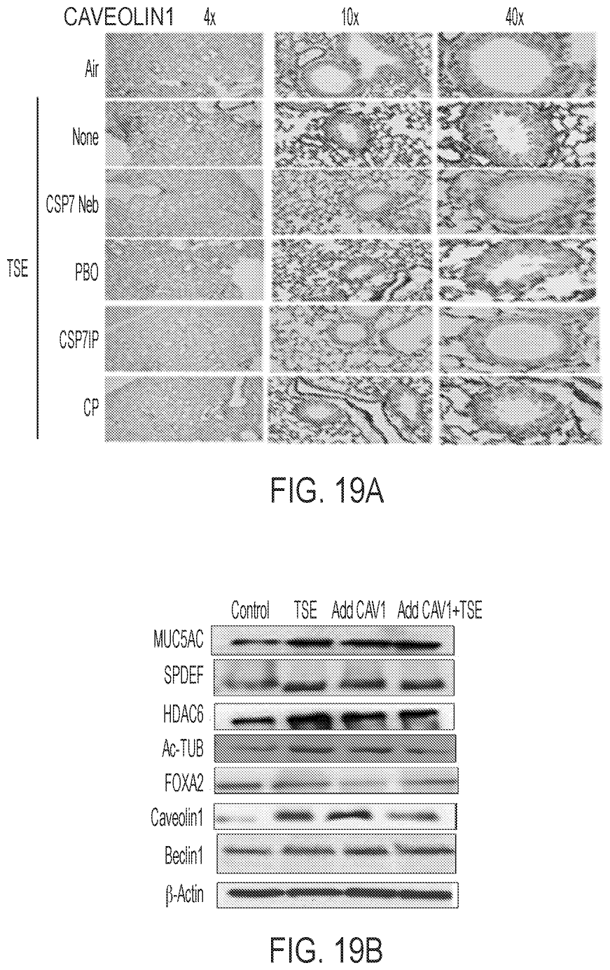

Increased Caveolin1 expression in AECs isolated from NL treated with TSE were observed, and this was reversed by CSP7. Histological analysis of lung sections showed increased expression of Caveolin1 in sections of 20 weeks TSE WT mice, which was overcome in CSP7 (Neb and IP) treated WT mice (FIG. 19A). Western blot analysis revealed that the overexpression of caveolin-1 induced in AECs by transduction of adenoviral vector expressing caveolin-1 caused a marked increase MUCSAC, HDAC6, SPDEF, FOXA3, and Caveolin-1 and a decrease in FOXA2 and Ac-Tub (cilia). Immunoblot experiments were done to investigate CSP7 suppression of the over-expression of caveolin (FIG. 19B). Interestingly, CSP7 can mitigate the mucus hypersecretion and cilia disassembly by inhibiting the role of overexpressed caveolin1.

### Example 17

### Role of p53 and PAI-1 in TSE Induced Mucin Hypersecretion and Cilia Dysfunction in Mouse Model

To determine whether lung epithelial injury due to TSE induced p53 and if p53 played a pivotal role in the induction of PAI-1 expression in vivo, WT mice were exposed to ambient air or passive TS for 20 weeks. PAI-1 was analyzed in bronchial alveolar lavage (BAL) fluids and lung homogenates, whereas p53 was analyzed in lung homogenates. To determine whether CSP7 inhibited p53 and PAI-1 expression induced by TSE, WT mice were injected with (or without CSP7 or CP, 4 weeks after initiation of passive TSE. At the end of 20 weeks, BAL fluids and lung homogenates were analyzed for changes in p53 and PAI-1. Consistent with the outcomes of AECs in vitro, CSP7 treatment of mice in vivo significantly suppressed the expression of p53 and PAI-1.

To investigate the role of p53 and PAI-1 in TSE induced mucin hypersecretion and cilia dysfunction, p53^{-/-} and PAI-1^{-/-} mice (n=10/group) were kept in ambient air or TSE for 4 hour/day 5 days a week as described. Histological analysis of lung sections also showed increased expression of MUCSAC in the lung section of TSE (20 wk) WT mice, which was suppressed in WT mice kept in ambient air, and in TSE p53^{-/-} and PAI-1^{-/-} mice.

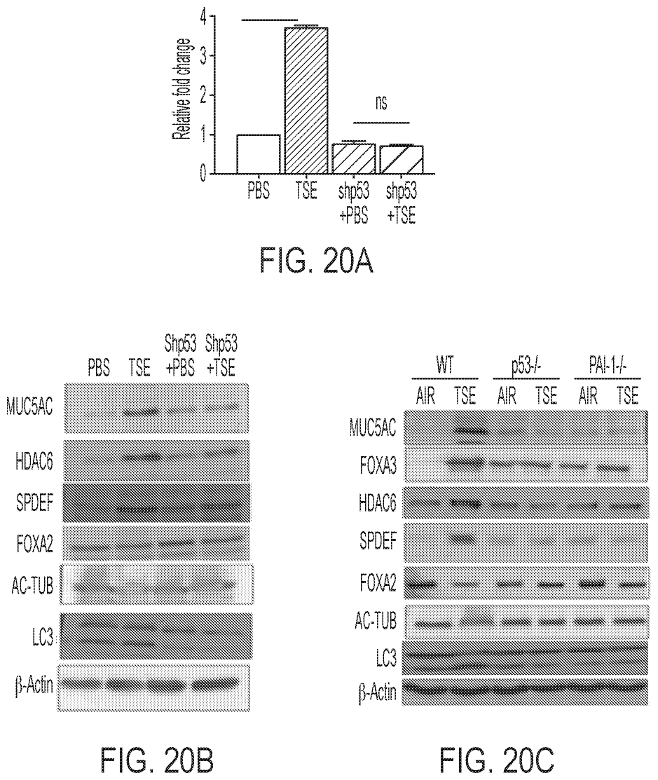

Western blot images showed increased Mucin related gene expression in the lung homogenates of TSE (20 weeks) WT mice, but resistance to this effect in p53^{-/-} and PAI-1^{-/-} (FIG. 20B-C). increased MUC5AC mRNA and protein expression in TSE-treated AECs, which was absent in TSE treated AECs transduced with Lvp53 shRNA. Thus mucus associated protein and autophagy markers were elevated in expression in TSE treated animals and diminished in AECs that had been transduced with Lvp53 shRNA(FIG. 20A).

### Example 18

### Mechanism of CSP7 Attenuation of Mucus Hypersecretion and Ciliary Disassembly: AECs from Subjects with COPD have Reduced PP2A Signalling

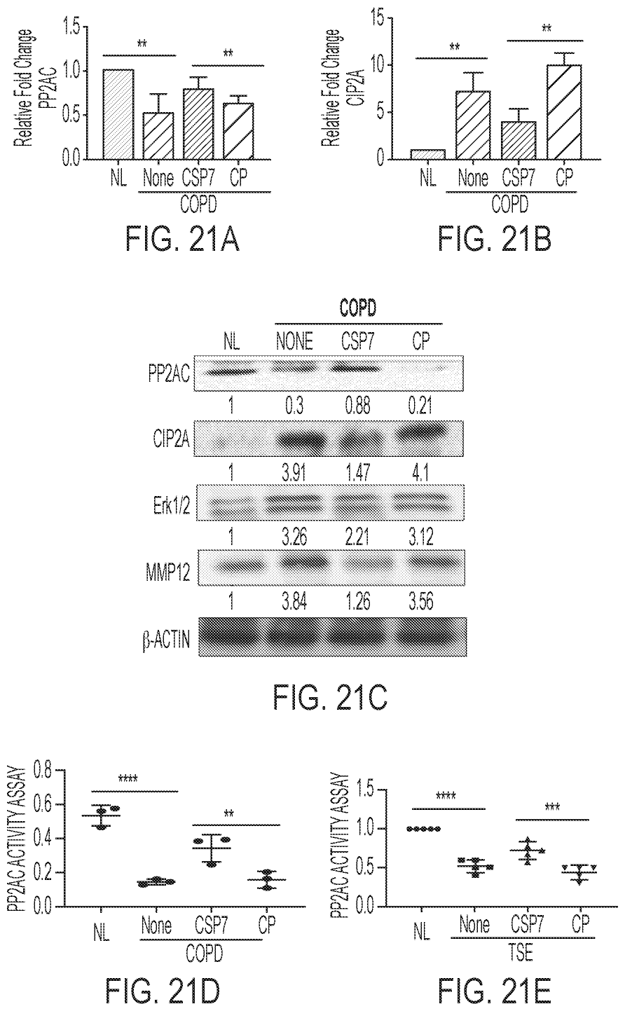

Protein phosphatase 2A (PP2A) activation is altered in emphysema lung samples. Therefore PP2A activity levels were examined in AECs isolated from NL and from subjects with COPD. PP2A activity was significantly decreased in AECs from subjects with COPD. PP2A activity influences ERK phosphorylation, so the loss of PP2A activity was further examined by investigating ERK phosphorylation. Increased ERK expression was found in AECs from subjects with COPD, as confirmed by Western blot. Expression of PP2AC was also observed. Phosphorylation of PP2Ac in AECs from COPD patients compared with those from NL, indicating an alternate cause for decrease of PP2AC in the airway epithelium of subjects with COPD (FIGS. 12A&C). CIP2A is an endogenous inhibitor of PP2AC. The expression CIP2A mRNA and protein levels were therefore investigated and were increased in COPD patient AECs compared to levels from NL (FIGS. 21B&C). Increased CIP2A gene expression and protein levels in subjects with COPD was concluded to be a likely a major cause of reduced PP2AC activity in COPD. CIP2A expression was increased in AECs isolated from subjects with COPD, which decreased PP2A activity and thus increased MMP12 expression and secretion. When CIP2A was inhibited by CSP7, increased activity of PP2AC was observed in COPD AECs. The increased PP2AC activity was further confirmed by a downstream decrease in ERK phosphorylation.

The proteases that are linked to the development of COPD and are regulated by PP2AC and MMP12 were investigated. CIP2A expression was increased in COPD AECs, which had decreased PP2AC activity and, thus, increased MMP12 secretion. The relative gene expression of MMP12 was decreased in NL AECs and from COPD AECs treated with CSP7. Therefore, CSP7 mitigate the effect on PP2AC, ERK, and MMP12 in COPD (FIG. 21C). COPD lung tissues exposed to CSP7 ex vivo had reduced PP2A signaling. Serine-threonine phosphatase activity for PP2A was determined for each individual and represented as picomoles of phosphate liberated per minute on the y-axis (FIG. 21D). WT mice (n=10/group) were kept in ambient air or exposed to TS for 4 h/days 5 day a week as. After 16 weeks, TSE WT mice were left untreated (None) or exposed to formulated CSP7 (5.8 mg) in 30 ml of PBS containing lactose monohydrate (154 mg) or placebo (Pbo) alone 2 h daily 5 d a week for 4 weeks using a Neb tower, or IP injected with 1.5 mg/kg of CSP7 or CP daily 5 d a week for 4 weeks, TSE exposure reduced PP2A signaling and was reversed by CSP7. Serine-threonine phosphatase activity for PP2A was determined for each individual and represented as picomoles of phosphate liberated per minute on the y-axis (FIG. 21E). The inventors conclude that the above represents an important mechanism by which CSP7 attenuates the effect of mucus hypersecretion and ciliary disassembly.

### Discussion of Examples 11-18

The chronic airflow limitation of COPD is caused by a mixture of small airway disease and pulmonary emphysema, usually due to significant exposure to noxious particles or gases. TSE is the most common identifiable risk factor for COPD, with smokers known to have a higher COPD mortality rate than non-smokers (Kim, V. et al., PLoS One. 10(2): e0116108, 2015). Pulmonary emphysema is believed to result from epithelial cell death caused by smoking; therefore, COPD research has been substantially devoted to programmed cell death. In COPD patients, airway epithelium undergoes remodeling, leading to hyperplasia and metaplasia of airway cells, including goblet cells. Goblet cell hyperplasia and hypertrophy is consistently found in the large airways of smokers with airflow obstruction (Saetta M et al., Am J Respir Crit Care Med. 161:1016-21, 2000; Innes A L et al., Chest. 130:1102-8, 2006). Such changes to goblet cells results in mucus overproduction, hypersecretion, and, ultimately, mucus accumulation in the airway lumen with serious pathological outcomes (Ramos F L et al., Int J Chron Obstruct. Pulm. Dis. 9:139-50, 2014. Excessive production is a consequence of increased synthesis and secretion of mucins and is often associated with increase in number of goblet cells. Based on this and the knowledge that smoking induces metaplasia of MUCSAC-positive cells in the airway epithelium of smokers, and increased expression of MUCSAC in the airway epithelium of smokers, targeting mucus hypersecretion alleviates COPD exacerbation.

Studies described herein investigated the effects of TSE on changes lung MUCSAC mRNA and protein expression, and mucus hypersecretion in a model of mouse emphysema and type I human AECs. The present findings include augmented expression of MUCSAC in COPD AECs as compared to normal AECs. Interestingly, small airways of humans (<2 mm lumenal diameter) and all intrapulmonary airways of mice, have few or no visible ‘mucous’ or ‘goblet’ cells under baseline conditions. In allergic inflammation or TSE, there is a rapid and dramatic increase mucous metaplasia or goblet cells (Evans, C et al., Am. J. Respir. Cell Mol. Biol. 31:382-94, 2004).

A developmental transcriptional regulator of goblet cell hypertrophy and hyperplasia is a sterile (?) alpha motif-pointed domain containing E26 transformation-specific like factor (SPDEF). SPDEF expression is increased in airways of COPD patients (Chen G et al., 2009; supra) and in long-term smokers (Chen G et al., 2014, supra). SPDEF upregulates several goblet cell differentiation genes, including that encoding forkhead box A3 (FOXA3) (Chen et al. 2014, supra) and endoplasmic reticulum protein anterior gradient protein 2 homolog. FOXA3 was sufficient to induce goblet cell metaplasia in vivo and in vitro. The present inventors' in vitro and in vivo studies showed that FOXA3 was sufficient to cause goblet cell metaplasia in airway epithelium. FOXA3 bound to and induced SPDEF, a gene required for goblet cell differentiation in the airway epithelium. Hence, the observed effects of FOXA3 on mucus related gene expression are likely mediated, at least in part, by the ability to induce SPDEF. However, FOXA3 directly bound to, and induced, AGR2 and MUCSAC that are critical for airway mucus production/goblet cell metaplasia (Williams O W et al., Am J Respir Cell Mol Biol 34:527-36, 2006; Schroeder B W et al., Am J Respir Cell Mol Biol 47:178-85, 2012), functioning independently of SPDEF to regulate these genes in human airway epithelial cells. Disruption of FOXA2 in respiratory epithelial cells caused airspace enlargement, pulmonary neutrophil infiltrates, and mucous metaplasia. SPDEF and MUCSAC have previously been shown to be highly expressed in bronchial epithelium of COPD patients (Chen et al., 2014, supra), which agrees with the present findings of increased expression of SPDEF, MUCSAC, and FOXA3 and decreased FOXA2 expression in COPD when compared to controls. In the present studies, treatment with CSP7 was found to reduce the effect of mucus hypersecretion-related genes.

Emerging evidence suggests that autophagy plays an important role in pulmonary diseases (Patel A S et al., PLoS One. 7:e41394, 2012; Ryter S W et al., Annu. Rev. Physiol. 74:377-401, 2012; Wu Y F et al., Autophagy. 2019 Jun. 16, https://doi.org/10.1080/15548627.2019.16285360). Prior reports demonstrated that autophagy was critical in mediating tobacco smoke-induced apoptosis of lung epithelial cells and contributed to development of emphysema (Chen Z H et al., PLoS One 3: e3316, 2008; Chen Z H et al., Proc Natl Acad Sci USA 107:18880-85, 2010.) As recently reported, exposure to particulate matter inactivated mTOR (a mechanistic target of rapamycin kinase), enhanced macroautophagy/autophagy, and impaired lysosomal activity in human bronchial epithelial cells and in mouse airway epithelium (Wu et al., 2019, supra). Moreover, autophagy also mediates TSE-induced cilia shortening and mitochondrial dysfunction in airway epithelium (Cloonan S M et al., Autophagy 10:5324, 2014; Lam H C et al., J Clin Invest 123:5212-30, 2013).

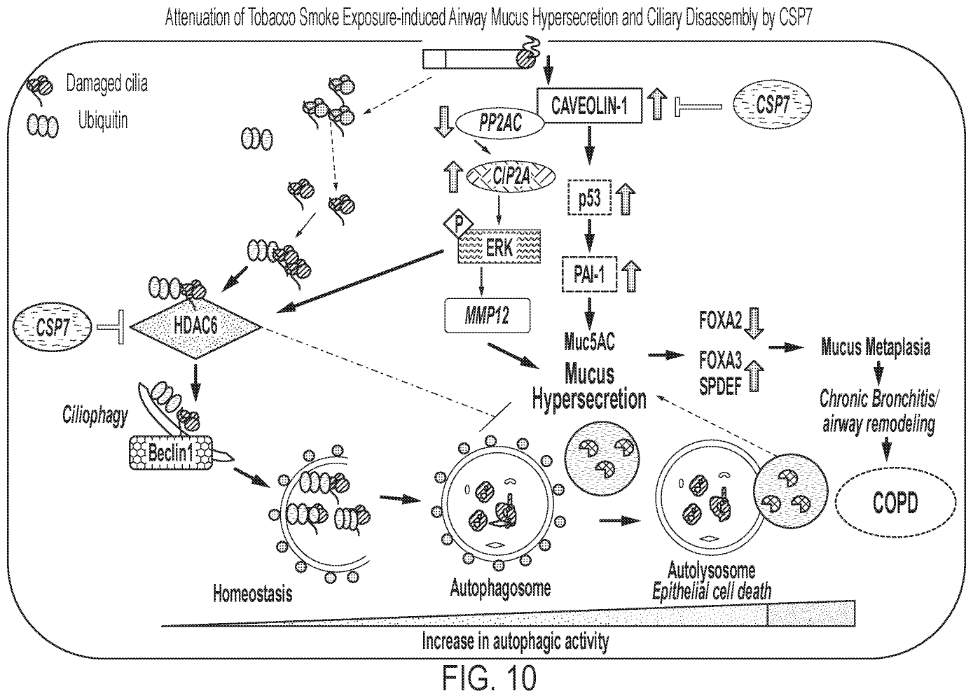

However, there is growing evidence that autophagy is a deleterious process that orchestrates various damage in airway epithelium during COPD pathogenesis. In the lungs, the “mucociliary escalator” acts as a primary innate defense mechanism, in which motile ciliated epithelial cells eliminate particles and pathogens trapped in mucus from the airways. Disruption of airway epithelial cell function as a result of TSE impairs mucociliary clearance (MCC). The mechanisms by which TSE-induced epithelial cell dysfunction leads to cilia shortening and altered airway function in vivo need further clarification. Among these mechanisms, cytosolic deacetylase HDAC6, which contains ubiquitin-binding and dynein-interacting domains, has emerged as a pleiotropic regulator of cellular function. HDAC6 controls diverse cellular processes through deacetylating and destabilizing microtubules (Pugacheva E N et al., Cell, 129:1351-63, 2007) facilitating retrograde transport of ubiquitinated proteins into aggresomes (Pandey U B, et al. Nature 447:859-63, 2007) and enhancing autophagosome-lysosome fusion Lee J Y, et al., EMBO J. 29:969-80, 2010). A role for HDAC6 has been found in motile cilia of the airways, in cellular responses to TSE exposure, and in COPD pathogenesis. This is illustrated schematically in FIG. 10. However, growing evidence indicates that HDAC6 recognizes ubiquitinated protein aggregates and delivers them to the autophagosome, a process dependent on the autophagy proteins LC3B and beclin 1. Ciliary proteins are delivered to the lysosome for degradation or recycling. In cases of chronic oxidative stress, ciliary proteins are degraded, resulting in a shortening of airway cilia that contributes to impaired mucociliary clearance. Interestingly the present inventors showed, in COPD and with TSE, increased HDAC6 and upregulation of autophagy markers leading to cilia shortening. They showed that HDAC6 increases, upregulation of autophagy molecules, and cilia shortening in COPD and co-TSE is reduced or attenuated by CSP7 treatment. Cilia components were shown to co-localize with autophagosomes based on Ac-Tub and LC3 co-localization. For the first time, interactions were found to occur between HDAC6 and MUCSAC in AECs in COPD, in TS-exposed AECs and in a murine emphysema model.

Caveolae are vesicular invaginations of the plasma membrane and the structural protein component of caveolae is caveolin-1. Caveolin-1 participates in signal transduction processes—acting as a scaffolding protein that concentrates, organizes and functionally regulates signaling molecules within caveolar membranes. TS, a source of oxidants, is an environmental hazard that causes pulmonary emphysema. Over-expression of caveolin-1 was enough to induce mucus hypersecretion and ciliary disassembly. Subsequently in the present studies mucus hypersecretion related genes and cilia were shown to be upregulated when caveolin-1 protein was overexpressed.

Insights into the molecular mechanism underlying free radical activation of the ataxia telangiectasia-mutated (ATM)-p53 pathway and a suggestion that caveolin-1 may be a novel therapeutic target for the treatment and/or prevention of pulmonary emphysema was described by Volonte D et al., J Biol Chem. 284:5462-6, 2009.

The present inventors and their colleagues previously demonstrated that tumor suppressor protein p53 augmented PAI-1 expression in AECs during TSE-induced lung injury. Chronic lung inflammation with elevated p53 and PAI-1 expression in AECs and increased susceptibility to and exacerbation of respiratory infections are all associated with COPD. (See Tiwari et al., 2016, supra). The present inventors and colleagues demonstrated that preventing p53 from binding to the endogenous PAI-1 mRNA in AECs by either suppressing p53 expression or blockading p53 interactions with the PAI-1 mRNA mitigated mucus hypersecretion and ciliary disassembly. A previous report elucidated the premature senescence of lung fibroblasts induced by oxidative stress which occurred by activation of ATM)/p53-dependent pathway following sequestration into caveolar membranes of the catalytic subunit of protein phosphatase 2A (PP2A-C), an inhibitor of ATM, by caveolin-1. A previous study demonstrated that loss of PP2A expression enhanced TS induced MMP1 and MMP9 expression (Wallace A M et al., Toxicol Sci 126:589-99, 2012). Although caveolae were known to be highly immobile and non-endocytic under normal conditions, in stress conditions or as a result of TSE, endocytosis occur via a caveolin-1-mediated process. PP2A activity was downregulated by chronic TSE and decreased in COPD, which subsequently modulated proteolytic responses. In addition, CIP2A is an inhibitor of PP2A. The present inventors showed that AECs from COPD subjects and active smokers had reduced PP2A activity as well as increased, CIP2A expression.

In the present studies, Caveolin 1 bound to PP2AC and was downregulated PP2AC activity, leading to increased CIP2A expression. Increased CIP2A led to phosphorylation of ERK, and secretion of MMP12. The caveolin 1-elevated p53 and PAI-1 expression in AECs and increased susceptibility to and exacerbation of respiratory infections are associated with COPD. Moreover, caveolin-1 expression was required for activation of the p53-PAI-1 pathway following stimulation with TSE extracts in vitro. Thus, according to this invention, caveolin-1 is a key player in a novel signaling pathway that links TSE to mucus hypersecretion and ciliary disassembly. A 7-mer peptide fragment of CSP, CSP7 (FTTFTVT, SEQ ID NO:1)) mitigated cilia shortening and impaired mucociliary clearance (MCC) by inhibiting caveolin-1. CSP7 also significantly downregulated phosphorylation of ERK, expression levels of MMP-12, and CIP2A. These findings provide not only new insights on how CSP7 regulates complex interrelationships between p53, PAI-1, autophagy and primary cilia. CSP7 is useful for treatment of the ciliopathy-associated mucus hypersecretion. This is the first discovery of
(a) CSP7 markedly reducing mucus hypersecretion and attenuating ciliary disassembly,
(b) understanding the underlying cellular and molecular mechanisms of caveolin's important role in TSE-associated cilia shortening and mucus hypersecretion by an endocytic process and
(c) the mitigation of these effects by CSP7.

According to the present invention a caveolin-1 scaffolding domain peptide CSP), and preferably, its biologically active peptide CSP7, is a new therapeutic agent for improving airway function during chronic lung diseases such as COPD by reversing, preventing or attenuating cilia shortening and impaired mucociliary clearance.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A method for
(A) blocking, reducing or attenuating:
(i) induction of p53 and PAI-1;
(ii) telomere dysfunction;
(iii) senescence and apoptosis in alveolar type II epithelial cells (A₂Cs);
(iv) mucus cell metaplasia;
(v) mucus hypersecretion mediated by overexpression of M5Ac or by IL-17A in airway epithelial cells (AECs);
(vi) expression of Forkhead box protein A3 (FOXA3);
(vii) expression of SAM-pointed domain containing ETS-like factor (SPEDF);
(viii) expression cancerous inhibitor of protein phosphatase 2A (CIP2A);
(ix) expression of histone deacetylase 6 (HDAC6);
(x) autophagic activity; or
(xi) disassembly, shortening or ciliopathy of airway cilia;
or
(B) increasing expression or upregulation of:
(xii) expression of forkhead box protein A2 (FOXA2);
(xiii) expression of catalytic unit of protein phosphatase-2A (PP2AC); comprising providing to A₂Cs or AECs in a subject an effective amount of a compound or composition that is:
(a) a peptide designated CSP7 the sequence of which is FTTFTVT (SEQ ID NO:1);
(b) an addition variant of (a) that includes 1-5 amino acids of additional sequence at the N- and/or C-terminus
(c) a covalently-modified chemical derivative of the peptide of (a) or (b),
(d) a peptide multimer of (a), (b) or (c);
(e) a deliverable peptide or polypeptide composition comprising the peptide, variant derivative or multimer of any of (a)-(d) bound to or associated with a delivery or translocation-molecule or moiety;
wherein said variant, chemical derivative or multimer has at least 20% of the biological or biochemical activity of said CSP7 in an in vitro or in vivo assay.

2. The method of claim 1 that results in a reduction of lung inflammation in said subject.

3. The method of claim 1 wherein the compound is CSP7 (FTTFTVT, SEQ ID NO:1).

4. The method of claim 1 wherein the peptide multimer comprises at least two monomers, each monomer being said CSP7 peptide, said addition variant or said chemical derivative, which multimer:
(a) has the formula P¹ₙ wherein
(i) P¹ is said peptide, variant or chemical derivative, and
(ii) n=2-5, or
(b) has the formula (P¹-Xₘ)ₙ-P², wherein
(i) each of P¹ and P² is, independently, the peptide, variant or chemical derivative,
(ii) each of P¹ and P² is the same or different peptide, variant or derivative
(iii) X is C₁-C₅ alkyl, C₁-C₅ alkenyl, C₁-C₅ alkynyl, C₁-C₅ polyether containing up to 4 oxygen atoms;
(iv) m=0 or 1; and
(v) n=1-7, or
(c) has the formula (P′-Glyn)n-P², wherein:
(i) each of P¹ and P² is, independently, said peptide, variant or derivative,
(ii) each of P¹ and P² is the same or different peptide or variant or derivative;
(iii) z=0-6; and
(iv) n=1-25.

5. The method of claim 4, wherein said peptide multimer comprises peptide monomers each of which is the CSP7 peptide FTTFTVT (SEQ ID NO:1).

6. The method of claim 1, wherein the peptide, addition variant, chemical derivative, multimer, or deliverable peptide or polypeptide is provided in vivo with a pharmaceutically acceptable carrier or excipient.

7. A method for treating a mammalian subject having an inflammatory lung disease or condition selected from the group consisting of COPD, emphysema, severe asthma, α1-anti-trypsin deficiency, cystic fibrosis, bronchiectasis, sarcoidosis, bronchiolitis obliterans, lung allograft fibrogenesis and lung transplant rejection, comprising administering to a subject in need thereof and effective amount of
(a) a pharmaceutical composition comprising a compound or composition selected from the group consisting of:
(i) a peptide designated CSP7 the sequence of which is FTTFTVT (SEQ ID NO:1);
(ii) an addition variant of (i) that includes 1-5 amino acids of additional sequence at the N- and/or C-terminus;
(iii) a covalently-modified chemical derivative of the peptide of (i) or (ii),
(iv) a peptide multimer of (i), (ii) or (iii); and
(v) a deliverable peptide or polypeptide composition comprising the peptide, variant, derivative or multimer of any of (i)-((iv) bound to or associated with a delivery or translocation-molecule or moiety;
wherein said addition variant, chemical derivative or multimer has at least 20% of the biological or biochemical activity of said CSP7 in an in vitro or in vivo assay, and
(b) a pharmaceutically acceptable carrier or excipient.

8. The method of claim 7, wherein the compound is CSP7 (FTTFTVT, SEQ ID NO:1).

9. The method of claim 7, wherein the peptide multimer comprises at least two monomers, each monomer being said CSP7 peptide, said addition variant or said chemical derivative, which multimer:
(a) has the formula P¹ₙ wherein
(i) P¹ is said peptide, variant or chemical derivative, and
(ii) n=2-5, or
(b) has the formula (P¹-Xₘ)ₙ-P², wherein
(i) each of P¹ and P² is, independently, the peptide, variant or chemical derivative,
(ii) each of P¹ and P² is the same or different peptide, variant or derivative
(iii) X is C₁-C₅ alkyl, C₁-C₅ alkenyl, C₁-C₅ alkynyl, C₁-C₅ polyether containing up to 4 oxygen atoms;
(iv) m=0 or 1; and
(v) n=1-7, or
(c) has the formula (P¹-Gly_{z})ₙ-P², wherein:
(i) each of P¹ and P² is, independently, said peptide, variant or derivative,
(ii) each of P¹ and P² is the same or different peptide or variant or derivative;
(iii) z=0-6; and
(iv) n=1-25.

10. The method of claim 9, wherein the peptide monomer is CSP7 (FTTFTVT, SEQ ID NO:1).

11. The method of claim 7, wherein the deliverable peptide or polypeptide of (a)(v) comprises the delivery or translocation molecule or moiety selected from the group consisting of
(A) HIV-TAT protein or a translocationally active derivative thereof;
(B) penetratin having the sequence RQIKIWFQNRRMKWKK (SEQ ID NO:6);
(C) a penetratin variant W48F having the sequence RQIKIFFQNRRMKWKK (SEQ ID NO:7);
(D) a penetratin variant W56F having the sequence RQIKIWFQNRRMKFKK, SEQ ID NO:8);
(E) a penetratin variant having the sequence RQIKIWFQNRRMKFKK, SEQ ID NO:9);
(F) transportan having the sequence GWTLNSAGYLLGKINLKALAALAKKIL (SEQ ID NO:10);
(G) herpes simplex virus protein VP22 or a translocationally-active homologue thereof from a different herpes virus such as MDV protein UL49; and
(H) Pep-1, having the sequence KETWWETWWTEWSQPKKKRKV (SEQ ID NO:11).

12. The method of claim 7, wherein the pharmaceutical composition is formulated for injection or lung instillation.

13. The method of claim 8, wherein the pharmaceutical composition is formulated for injection or lung instillation.

14. The method of claim 9, wherein the pharmaceutical composition is formulated for injection or lung instillation.

15. The method of claim 12, wherein the pharmaceutical composition is formulated for lung instillation.

16. The method of claim 7, wherein the pharmaceutical composition is formulated for oral, parenteral, topical, transdermal, intravaginal, intrapenile, intranasal, intrabronchial, intracranial, intraocular, intraaural or rectal administration.

17. The method of claim 1 wherein the cells are human cells.

18. The method of claim 6, wherein the cells are human cells.

19. The method of claim 1, wherein the subject is a human.

20. The method of claim 6, wherein the subject is a human.

21. The method of claim 7, wherein the subject is a human.

22. The method of claim 15, wherein the subject is a human.

23-24. (canceled)
